(19) United States
(12) Patent Application Publication
(10) Pub. No.: US 2007/0059833 A1
(43) Pub. Date: Mar. 15, 2007

(54) Use of Nucleases to Improve Viability and Enhance Transgene Expression in Transfected Cells

(75) Inventors: Linhong Li, North Potomac, MD (US); Linda N. Liu, Clarksville, MD (US); Cornell Allen, Woodlawn, MD (US); Rama Shivakumar, Bethesda, MD (US); James Brady, Germantown, MD (US)

Correspondence Address: FULBRIGHT & JAWORSKI L.L.P., 600 CONGRESS AVE., SUITE 2400, AUSTIN, TX, 78701, US

(73) Assignee: MAXCYTE, INC.

(21) Appl. No.: 11/470,906

(22) Filed: Sep. 7, 2006

Related U.S. Application Data
(60) Provisional application No. 60/714,620, filed on Sep. 7, 2005.

(51) Int. Cl. C12N 15/87

(52) U.S. Cl. 435/459

(57) ABSTRACT

The present invention concerns methods and compositions for improving viability and transgene expression in transfected cells. In one embodiment, the present invention provides a method for increasing the viability of a transfected cell, the method comprising: transfecting a cell with a nucleic acid sequence; and contacting the transfected cell with a nuclease in a manner effective to enhance the viability of the transfected cell.

The present application claims the benefit of U.S. Provisional Patent Application Ser. No. 60/714,620, filed Sep. 7, 2005, the entire disclosure of which is specifically incorporated herein by reference.

### BACKGROUND OF THE INVENTION

A. Field of the Invention

The present invention relates generally to the field of molecular biology. More particularly, it concerns methods and compositions for improving viability and transgene expression in transfected cells.

B. Description of Related Art

The transfection mechanism of gene delivery systems involves the passage of DNA molecules through various biological barriers. Transfection procedures must in some way permeabilize the cell membrane to permit the transfer of DNA molecules into the target cell. This permeabilization must be temporary and reversible if the transfected cell is to survive. Although numerous transfection methods are routinely used to transfect cells, necrosis and apoptosis of cells subject to transfection protocols can be obstacles to achieving efficient transfection.

Transfection methods that can provide improved transfected cell viability and efficient transgene expression would be advantageous in numerous applications including, for example, the production of recombinant cells and proteins that have therapeutic, industrial, or research uses. The present invention provides such methods.

### SUMMARY OF THE INVENTION

In one embodiment, the present invention provides a method for increasing the viability of a transfected cell, the method comprising: transfecting a cell with a nucleic acid sequence; and contacting the transfected cell with a nuclease in a manner effective to enhance the viability of the transfected cell. In certain embodiments, the viability of the transfected cell is enhanced relative to a control transfected cell not contacted with the nuclease.

In another embodiment, the present invention provides a method for increasing the transfection efficiency in a population of transfected cells, the method comprising: transfecting the cells with a nucleic acid sequence; and contacting the transfected cells with a nuclease in a manner effective to increase the transfection efficiency in the population of transfected cells. In certain embodiments, the transfection efficiency in the population of transfected cells is increased relative to the transfection efficiency in a control population of transfected cells not contacted with the nuclease.

As used herein, “increasing the transfection efficiency” in a population of cells may be defined as increasing the percentage of viable, transfected cells in the population. “Increasing the transfection efficiency” in a population of cells may also be defined as increasing the percentage of transfected cells in the population, and/or increasing the expression level of a transgene in the transfected cells in the population. As used herein, “enhancing transgene expression” means increasing the level of transgene expression and/or prolonging transgene expression.

The present invention may be used to improve the viability of, or enhance the transfection efficiency in, any type of cell or population of cells. In some embodiments the cell is a eukaryotic cell, such as a mammalian cell, insect cell, plant cell, or yeast cell. Examples of preferred mammalian cells include human, mouse, hamster, and rat cells. The cell may be a primary cell or an established cell line, such as a K562 cell, a 293T cell, or a Jurkat cell. The cell may be a cancer cell, such as a breast cancer cell, lung cancer cell, prostate cancer cell, ovarian cancer cell, brain cancer cell, liver cancer cell, cervical cancer cell, colon cancer cell, renal cancer cell, skin cancer cell, head & neck cancer cell, bone cancer cell, esophageal cancer cell, bladder cancer cell, uterine cancer cell, lymphatic cancer cell, stomach cancer cell, pancreatic cancer cell, testicular cancer cell, or leukemia cell (e.g., AML, ALL, CML, or CLL cells). In other embodiments the cell is a prokaryotic cell, such as a bacteria cell. The transgene may be integrated into the genomic DNA of the host cell or it may be extrachromosomal. In certain aspects of the invention, the cell is a germ cell, such as a spermatozoa or an unfertilized egg cell.

Any nuclease may be used in connection with the present invention. As used herein, “nuclease” refers to any enzyme capable of cleaving or hydrolyzing nucleic acids. A nuclease may be an endonuclease or an exonuclease. An endonuclease is any of a group enzymes that catalyze the hydrolysis of bonds between nucleic acids in the interior of a DNA or RNA molecule. An exonuclease is any of a group of enzymes that catalyze the hydrolysis of single nucleotides from the end of a DNA or RNA chain. A nuclease that specifically catalyzes the hydrolysis of DNA may be referred to as a deoxyribonuclease or DNase, whereas nuclease that specifically catalyses the hydrolysis of RNA may be referred to as a ribonuclease or an RNase. Those of ordinary skill in the art will be able to select an appropriate nuclease depending on the characteristics of the nucleic acid sequence that is being transfected in to the cell. For example, where the nucleic acid sequence is an RNA, then a ribonuclease should be used. Where the nucleic acid sequence is a DNA, then a deoxyribonuclease should be used. If a restriction endonuclease is used to digest DNA, the choice of enzyme can be based on the cutting frequency of the enzyme and the number of recognition sequences in the transfected DNA. A combination of nucleases may be used to achieve the desired amount of nucleic acid degradation. In a preferred embodiment the nuclease is DNaseI.

It is also contemplated that non-enzymatic nucleic acid hydrolyzing agents may be used in the methods of the present invention. Cerium(IV)/ethylene-diamine-N,N,N′,N′-tetraacetate (Ce(IV)/EDTA) is an example of a non-enzymatic nucleic acid hydrolyzing agent (Kitamura et al., 2002; Yamamoto et al., 2003; Yamamoto et al., 2004). Non-enzymatic nucleic acid hydrolyzing agents may be used in the methods and compositions described herein either in place of the nucleases or in addition to the nucleases. Nucleases and non-enzymatic nucleic acid hydrolyzing agents may be referred to collectively as “nucleic acid hydrolyzing agents” or “nucleic acid digesting agents.”

In certain aspects of the invention, the nuclease will be added to the transfection buffer, the culture medium, or both. The concentration of nuclease will vary depending on conditions such as the cell type, the particular nuclease being used, the nucleic acid concentration, and the composition of the buffer or culture medium in which the reaction occurs. Optimizing such reaction conditions is routine to those of ordinary skill in the art.

The nuclease should be mixed with the cell after transfection. The nuclease may be added immediately post-transfection. However, it is not necessary to add the nuclease immediately post-transfection, as beneficial results can be obtained when the nuclease is administered up to 2 hours or more post-transfection. In some embodiments, the transfected cell is contacted with the nuclease between 0-120 minutes after transfection. In some embodiments, the transfected cell is contacted with the nuclease between 0-60 minutes after transfection. In certain embodiments the transfected cell is contacted with the nuclease between 0-20 minutes after transfection or between 20-60 minutes after transfection. The nuclease may be added at up to about 1, 5, 10, 15, 20, 25, 30, 35, 40, 45, 50, 55, 60, 70, 80, 90, 100, 110, or 120 minutes post-transfection.

The amount of time the cell is incubated with a nuclease can also vary. The amount of time can very depending on reaction conditions such as temperature, pH, enzyme concentration, and nucleic acid concentration. It is routine for those of ordinary skill in the art to optimize such reaction conditions for digesting nucleic acids. In certain embodiments, the cell is incubated with the nuclease for up to about 5, 10, 15, 20, 25, 30, 35, 40, 45, 50, 55, or 60 minutes. It should be noted, however, that nucleases generally are not toxic to cells. Thus, it is not required that the nuclease be removed following incubation with the transfected cells. For example, transfected cells could be incubated with DNase I for a period of time (e.g., 20 minutes) and then transferred to the culture media without removing the DNase I.

The present invention may be used in connection with the transfection of any nucleic acid sequence into a cell. The nucleic acid may be a DNA or a RNA. In certain embodiments, the cell and the nucleic acid sequence are incubated together prior to transfection. In certain aspects the cell and the nucleic acid sequence are incubated for at least 0, 1, 2, 5, 10, 15, 20, 25, 30, 40, 50, 60, or more minutes prior to transfection. In one aspect of the invention, the cell and the nucleic acid sequence are incubated for between about 0-20 minutes prior to transfection.

The present invention may be used to improve the viability and transfection efficiency in cells transfected with any size of nucleic acid molecule; however, the present invention is particularly advantageous for improving the viability and transfection efficiency in cells transfected with large nucleic acid sequences. In certain embodiments of the invention, the nucleic acid sequence is greater than or equal to about 4, 5, 6, 7, 8, 9, 10, 11, 12, 13, 14, 15, 16, 17, 18, 19, or 20 kilobases (kb) in length.

The present invention may be used to improve the viability and transfection efficiency in cells transfected with a variety of nucleic acid concentrations. In certain embodiments, the concentration of the nucleic acid sequence is at least about 5 μg/ml, 50 μg/ml, 100 μg/ml, 150 μg/ml, 200 μg/ml, 250 μg/ml, 300 μg/ml, 350 μg/ml, 400 μg/ml, 450 μg/ml, 500 μg/ml, 550 μg/ml, 600 μg/ml, 650 μg/ml, 700 μg/ml, 750 μg/ml, or 800 μg/ml. In other aspects of the invention the concentration of the nucleic acid sequence is between about 5-25 μg/ml, 25-50 μg/ml, 50-100 μg/ml, 100-150 μg/ml, 150-200 μg/ml, 200-250 μg/ml, 250-500 μg/ml, 5-800 μg/ml, 200-800 μg/ml, 250-800 μg/ml, 400-800 μg/ml, 500-800 μg/ml, or any range derivable therein.

In one embodiment, the nucleic acid sequence is a sequence that is not transcribed or translated, but that has properties useful in itself. For example, the nucleic acid sequence may be an aptamer. The aptamer may be a DNA or RNA aptamer. The nucleic acid sequence may be, for example, a non-protein coding RNA, such as a ribosomal RNA, tRNA, splicosomal RNA, antisense RNA, siRNA, or mRNA.

In one embodiment, the nucleic acid sequence is an expression vector. The expression vector may be, for example, a plasmid. In some embodiments, the expression vector encodes a peptide, polypeptide, or protein. In other embodiments, the expression vector encodes a non-protein coding RNA, such as a ribosomal RNA, tRNA, splicosomal RNA, antisense RNA, siRNA, or mRNA.

In some embodiments of the invention, the method further comprises culturing the transfected cells under conditions conducive to the expression of the peptide, polypeptide, protein, or the non-protein coding RNA. In yet other embodiments, the method further comprises isolating the peptide, polypeptide, protein, or the non-protein coding RNA from the cell or from the culture medium.

In some embodiments, the expression vector encodes a cytosolic protein, a membrane protein, or a secreted protein. The protein may be a therapeutic protein. A “therapeutic protein” is a protein that can be administered to a subject for the purpose of treating or preventing a disease. Examples of classes of therapeutic proteins include tumor suppressors, inducers of apoptosis, cell cycle regulators, immuno-stimulatory proteins, toxins, cytokines, enzymes, antibodies, inhibitors of angiogenesis, angiogenic factors, growth factors, metalloproteinase inhibitors, hormones, or peptide hormones. The therapeutic protein may be isolated from the cell from which it was produced prior to administering it to a subject. Alternatively, the transfected cell expressing the therapeutic protein may be administered to a subject.

An “immuno-stimulatory protein” is a protein involved in the activation, chemotaxis, or differentiation of immune cells. Examples of classes of immuno-stimulatory proteins include thymic hormones, cytokines, and growth factors. Thymic hormones include, for example, prothymosin-α, thymulin, thymic humoral factor (THF), THF-γ-2, thymocyte growth peptide (TGP), thymopoietin (TPO), thymopentin, and thymosin-α-1. Examples of cytokines include, IL-1α, IL-1β, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, leukocyte inhibitory factor (LIF), IFN-α, IFN-β, IFN-γ, TNF, TNF-α, TGF-β, G-CSF, M-CSF, and GM-CSF. Other immuno-stimulatory proteins include B7.1 (CD80), B7.2 (CD86), ICAM-1 (CD54), VCAM-1, LFA-1, VLA-4, CD40, and CD40L (CD154).

Examples of other proteins contemplated by the present invention include developmental proteins such as adhesion molecules, cyclin kinase inhibitors, Wnt family members, Pax family members, Winged helix family members, Hox family members, cytokines/lymphokines and their receptors, growth or differentiation factors and their receptors, neurotransmitters and their receptors; oncogenes such as ABLI, BLC1, BCL6, CBFA1, CBL, CSFIR, ERBA, ERBB, EBRB2, ETS1, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TAL1, TCL3, and YES; tumor suppressors such as p53, Rb, Rap1A, DCC, k-rev, BRCA1, BRCA2, zac1, p73, MMAC-1, ATM, HIC-1, DPC-4, FHIT, APC, DCC, PTEN, ING1, NOEY1, NOEY2, PML, OVCA1, MADR2, WT1, 53BP2, IRF-1, MADH4, MCC, NF1, NF2, RB1, TP53, and WT1; enzymes such as carbamoyl synthetase I, ornithine transcarbamylase, arginosuccinate synthetase, arginosuccinate lyase, arginase, ACP desaturases and hycroxylases, ADP-glucose pyrophorylases, ATPases, alcohol dehydrogenases, amylases, amyloglucosidases, catalases, cellulases, cyclooxygenases, decarboxylases, dextrinases, esterases, DNA and RNA polymerases, hyaluron synthases, galactosidases, glucanases, glucose oxidases, GTPases, helicases, hemicellulases, hyaluronidases, integrases, invertases, isomersases, kinases, lactases, lipases, lipoxygenases, lyases, lysozymes, pectinesterases, peroxidases, phosphatases, phospholipases, phophorylases, polygalacturonases, proteinases and peptideases, pullanases, recombinases, reverse transcriptases, topoisomerases, and xylanases; and hormones such as growth hormone, prolactin, placental lactogen, luteinizing hormone, follicle-stimulating hormone, chorionic gonadotropin, thyroid-stimulating hormone, leptin, adrenocorticotropin (ACTH), angiotensin I and II, β-endorphin, β-melanocyte stimulating hormone (β-MSH), cholecystokinin, endothelin I, galanin, gastric inhibitory peptide (GIP), glucagon, insulin, lipotropins, neurophysins, somatostatin, calcitonin, calcitonin gene related peptide (CGRP), β-calcitonin gene related peptide, hypercalcemia of malignancy factor, parathyroid hormone-related protein (PTH-rP), glucagon-like peptide (GLP-1), pancreastatin, pancreatic peptide, peptide YY, PHM, secretin, vasoactive intestinal peptide (VIP), oxytocin, vasopressin (AVP), vasotocin, enkephalinamide, metorphinamide, alpha melanocyte stimulating hormone (alpha-MSH), atrial natriuretic factor (ANF), amylin, amyloid P component (SAP-1), corticotropin releasing hormone (CRH), growth hormone releasing factor (GHRH), luteinizing hormone-releasing hormone (LHRH), neuropeptide Y, substance K (neurokinin A), substance P, and thyrotropin releasing hormone (TRH).

Other desirable gene products include fumarylacetoacetate hydrolase, phenylalanine hydroxylase, alpha-1 antitrypsin, glucose-6-phosphatase, low-density-lipoprotein receptor, porphobilinogen deaminase, factor VIII, factor IX, cystathione β-synthase, branched chain ketoacid decarboxylase, albumin, isovaleryl-CoA dehydrogenase, propionyl CoA carboxylase, methyl malonyl CoA mutase, glutaryl CoA dehydrogenase, insulin, β-glucosidase, pyruvate carboxylase, hepatic phosphorylase, phosphorylase kinase, glycine decarboxylase (also referred to as P-protein), H-protein, T-protein, Menkes disease copper-transporting ATPase, Wilson's disease copper-transporting ATPase, cytosine deaminase, hypoxanthine-guanine phosphoribosyltransferase, galactose-1-phosphate uridylyltransferase, galactokinase, UDP-galactose-4-epimerase, phenylalanine hydroxylase, glucocerbrosidase, sphingomyelinase, α-L-iduronidase, glucose-6-phosphate dehydrogenase, HSV thymidine kinase, human thymidine kinase, blood derivatives, growth factors, neurotransmitters or their precursors or synthetic enzymes, trophic factors (such as BDNF, CNTF, NGF, IGF, GMF, aFGF, bFGF, NT3, NT5, and the like), apolipoproteins (such as ApoAI, ApoAIV, ApoE, and the like), dystrophin or a minidystrophin, factor VII, factor VIII, factor IX, fibrin, fibrinogen, thrombin, cytosine deaminase, all or part of a natural or artificial immunoglobulin (Fab, ScFv, and the like), anti-thrombotic genes (e.g., COX-1, TFPI); genes involved in angiogenesis (e.g., VEGF, aFGF, bFGF, FGF-4, FGF-5, thrombospondin, BAI-1, GDAIF, or their receptors), MCC, and mouse or humanized monoclonal antibodies.

The protein can also be an antigenic peptide or polypeptide capable of generating an immune response. Examples include polynucleotides encoding antigens such as viral antigens, bacterial antigens, fungal antigens or parasitic antigens. Virus targets include picomavirus, coronavirus, togavirus, flavivirus, rhabdovirus, paramyxovirus, orthomyxovirus, bunyavirus, arenvirus, reovirus, retrovirus, papovavirus, parvovirus, herpesvirus, poxvirus, hepadnavirus, and spongiform virus. Parasite targets include trypanosomes, tapeworms, roundworms, and helminthes. Also, tumor markers, such as fetal antigen or prostate specific antigen, may be targeted in this manner.

In one embodiment of the invention, the expression vector encodes one or more viral genes. The viral genes may be, for example, retroviral genes, lentiviral genes, alphaviral genes, adenoviral genes, or adeno-associated viral genes. In a preferred embodiment, the viral genes necessary to produce a non-replicating viral vector are provided on at least two different plasmids. A transgene of interest may also be provided on one of the plasmids for encapsulation in the non-replicating viral vector.

A cell may be transfected with two or more different nucleic acid sequences. For example, the cell may be transfected with 2, 3, 4, or more expression vectors, each encoding a different peptide, polypeptide, protein, or non-protein coding nucleic acid. As a further example, a cell may be transfected with 2, 3, 4, or more different inhibitory RNA molecules (e.g., siRNA or mRNA molecules).

The present invention may be used with any transfection method. Such methods include, for example, electroporation, calcium phosphate precipitation, liposome-mediated transfection, polymer-mediated transfection, viral transfection, and ballistic transfection. Those of skill in the art are familiar with these and other cell transfection methods. In some aspects of the invention, the transfection method is a method that does not require viral transfection.

In a preferred embodiment, the method of transfection is electroporation. Any method of transfecting cells by electroporation known in the art may be used in the present invention. The electroporation may be, for example, static electroporation, flow electroporation, variable flow electroporation, or streaming electroporation. In certain embodiments of the invention, the electroporation is performed at about 1.20 kV/cm, at about 1.33 kV/cm, or at about 1.50 kV/cm. In some embodiments of the invention, the electroporation is performed between about 0.50-5.00 kV/cm, 1.00-1.25 kV/cm, 1.25-1.50 kV/cm, 1.50-1.75 kV/cm, 1.50-5.00 kV/cm, 1.75-5.00 kV/cm, 2.00-5.00 kV/cm, 2.25-5.00 kV/cm, or any range derivable therein.

In certain aspects of the invention, the method of transfecting the cells comprises use of an electroporation device as described in U.S. patent application Ser. No. 10/225,446, incorporated herein by reference. Methods and devices for electroporation are also described in, for example, published PCT Application Nos. WO 03/018751 and WO 2004/031353; U.S. patent application Ser. Nos. 10/781,440, 10/080,272, and 10/675,592; and U.S. Pat. Nos. 5,720,921, 6,074,605, 6,773,669, 6,090,617, 6,485,961, 6,617,154, 5,612,207, all of which are incorporated by reference.

In one embodiment, the invention provides a method for increasing viability of a cell after electroporation, the method comprising: transfecting a cell with a nucleic acid sequence by electroporation; and contacting the cell with a nuclease after electroporation, wherein the viability of the cell after electroporation is increased as compared to the viability of a second cell not contacted with the nuclease after electroporation. In some embodiments, the method further comprises incubating the cell in electroporation buffer after electroporation. The cell may be incubated in the electroporation buffer for about 0-20 minutes or more. In certain aspects of the invention, the nuclease is added to the electroporation buffer during the incubation. In some embodiments, the method further comprises culturing the cell after electroporation. The cell may be cultured in any suitable culture medium. In certain aspects of the invention, the nuclease is added to the culture medium. In some embodiments, the nuclease is added to both the electroporation buffer and the culture medium. In certain embodiments, the cell is contacted with the nuclease between 0-60, 0-16, or 16-60 minutes after electroporation.

It is contemplated that any method or composition described herein can be implemented with respect to any other method or composition described herein.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A and 1B: Post electroporation DNase treatment improved viability of transfected Jurkat cells. Jurkat cells were transfected with a plasmid DNA carrying full-length cDNA encoding for the GPF marker gene at either 1.2 or 1.33 kv/cm. Cells without electroporation (0 v/cm) served as controls. The processed cells were either treated with DNase (solid column) or not treated (grey column). FIG. 1A shows the percentage of viable cells measured by FACS analysis of propidium iodine negative cells and FIG. 1B shows the total number of viable cells with or without DNase treatment.

FIG. 2: Post-electroporation DNase treatment enhanced transgene expression in transfected Jurkat cells. Jurkat cells were transfected with a plasmid DNA carrying full-length cDNA encoding for mIL4 at 1.2 or 1.33 kv/cm. Cells without electroporation (0 v/cm) served as controls. The processed cells were either treated with DNase (solid column) or not treated (grey column). The conditioned media was collected and analyzed at 48 hours post transfection for mIL4 production using a commercially available mIL4 ELISA kit (R&D System). FIG. 2 shows that populations of cells treated with DNase following electroporation-mediated transfection at both electrical pulse levels produced more mIL-4 than the cells that were not treated with DNase. Greater enhancement was observed when using higher field strength, 1.33 kv/cm.

FIG. 3: Post-electroporation DNase treatment allowed cells to tolerate higher DNA concentrations. Jurkat cells were transfected with the GFP marker gene plasmid at various DNA concentrations up to 200 μg/mL (70 nM). The transfected Jurkat cells were either treated (+) or not treated (−) with DNase and examined by FACS analysis for GFP expression and cell viability at 24 hours post transfection. A significantly higher percentage of viable cells were observed when Jurkat cells were treated with DNase following electroporation in the presence of higher concentrations of DNA.

FIGS. 4A and 4B: Post-electroporation DNase treatment increased the percentage of GFP+ Cells. A significantly greater number of GFP+ cells were observed when Jurkat cells were treated with DNase following electroporation in the presence of higher concentrations of DNA (FIG. 4A). The mean fluorescence intensity of GFP-expressing cells was similar with or without DNase treatment (FIG. 4B).

FIG. 5: Effect of DNase treatment at various time points throughout transfection. Hematopoeitic K562 cells were transfected with a plasmid DNA carrying full-length cDNA encoding for mIL12. DNase was either preadded to the DNA-cell mixture (−2 min), or during (0 min), or 1, 2, 4, 8, 16 mins post electroporation. Cells without DNase treatment (∞ min) served as controls. All cell viability was analyzed at 4 hours post transfection by FACS examination of propidium iodine (PI) stained cells and plotted out the PI negative cell population. Total cell number and mIL-12 production were analyzed at 48 hours post transfection.

FIG. 6: Post-electroporation DNase treatment improved membrane recovery. K562 cells were electroprated with the GFP marker gene plasmid. The transfected K562 cells were either treated or not treated with DNase. Cell samples were taken out and stained with trypan blue immediately after EP before DNase treatment (0 min), or 4, 7, 12, 180 minutes post EP. Significantly fewer trypan blue positive cells were observed when the transfected cells were treated with DNase (black solid column) suggesting the DNase helped membrane rehealing after transfection.

FIG. 7: Post-electroporation DNase treatment improved viability of 293T cells. Microscopic images taken of cultured 293T cells 16 hours post-EP with the plasmid pGAG-Endo-IRES-Angio show significantly more viable cells in the DNase treated population than in controls.

FIG. 8: Post-electroporation DNase treatment increased the total number of viable cells and mIL-12 production in 293T Cells. 293T cells were transfected with the pGAG-mILL2 plasmid and then either treated (sold black column) or not treated (grey column) with DNase. Total cell number and mIL-12 production were analyzed at 24 hours post transfection. Transfected 293T cells treated with DNase exhibited greater viability and protein production than controls 24 hours post-EP.

FIG. 9: Post-electroporation DNase treatment increased viral vector production. Transfected 293T cells treated with DNase following electroporation exhibited significantly increased lentiviral vector production efficiency than controls.

### DESCRIPTION OF ILLUSTRATIVE EMBODIMENTS

### A. Nucleases

The present invention provides methods for improving the viability and transgene expression from transfected cells by treating the cells post-transfection with a nuclease. Nucleases are enzymes that hydrolyze nucleic acids. Nucleases may be classified as endonucleases or an exonucleases. An endonuclease is any of a group enzymes that catalyze the hydrolysis of bonds between nucleic acids in the interior of a DNA or RNA molecule. An exonuclease is any of a group of enzymes that catalyze the hydrolysis of single nucleotides from the end of a DNA or RNA chain. Nucleases may also be classified based on whether they specifically digest DNA or RNA. A nuclease that specifically catalyzes the hydrolysis of DNA may be referred to as a deoxyribonuclease or DNase, whereas a nuclease that specifically catalyses the hydrolysis of RNA may be referred to as a ribonuclease or an RNase. Some nucleases are specific to either single-stranded or double-stranded nucleic acid sequences. Some enzymes have both exonuclease and endonuclease properties. In addition, some enzymes are able to digest both DNA and RNA sequences. The term “nuclease” is used herein to generally refer to any enzyme that hydrolyzes nucleic acid sequences.

According to the methods of the present invention, the nuclease may be added to the transfected cells immediately following transfection or up to several minutes to several hours post-transfection. The nuclease may be added to the same buffer in which the transfection occurred and/or it may be added to the medium in which the cells are cultured following transfection.

Optimal reaction conditions vary among the different nucleases. The factors that should be considered include temperature, pH, enzyme cofactors, salt composition, ionic strength, and stabilizers. Suppliers of commercially available nucleases (e.g., Promega Corp.; New England Biolabs, Inc.) provide information as to the optimal conditions for each enzyme. Most nucleases are used between pH 7.2 and pH 8.5 as measured at the temperature of incubation. In addition, most nucleases show maximum activity at 37° C.; however, a few enzymes require higher or lower temperatures for optimal activity (e.g., Taq I, 65° C.; Sma I, 25° C.). DNA concentration can also be a factor as a high DNA concentration can reduce enzyme activity, and DNA concentrations that are too dilute can fall below the Km of the enzyme and also affect enzyme activity. Where combinations of nucleases are used it may not always be possible to provide the optimal conditions for every enzyme in a single reaction. In these situations, conditions can be used in which all enzymes have an acceptable level of activity. If there are no conditions in which all of the enzymes can be used simultaneously, then the reactions can be performed sequentially. Those in the art are familiar with the use of nucleases and it is routine to adjust reaction conditions for particular applications.

Non-limiting examples of nucleases include, DNase I, Benzonase, Exonuclease I, Exonuclease III, Mung Bean Nuclease, Nuclease BAL 31, RNase I, S1 Nuclease, Lambda Exonuclease, RecJ, and T7 exonuclease. DNase I is an endonuclease that nonspecifically cleaves DNA to release di-, tri- and oligonucleotide products with 5′-phosphorylated and 3′-hydroxylated ends. DNase I acts on single- and double-stranded DNA, chromatin, and RNA:DNA hybrids. Exonuclease I catalyzes the removal of nucleotides from single-stranded DNA in the 3′ to 5′ direction. Exonuclease III catalyzes the stepwise removal of mononucleotides from 3′-hydroxyl termini of duplex DNA. Exonuclease III also acts at nicks in duplex DNA to produce single-strand gaps. Single-stranded DNA is resistant to Exonuclease III. Mung Bean Nuclease degrades single-stranded extensions from the ends of DNA. Mung Bean Nuclease is also an RNA endonuclease. Nuclease BAL 31 degrades both 3′ and 5′ termini of duplex DNA. Nuclease BAL 31 is also a highly specific single-stranded endonuclease that cleaves at nicks, gaps, and single-stranded regions of duplex DNA and RNA. RNase I is a single strand specific RNA endonuclease that will cleave at all RNA dinucleotide. S1 Nuclease degrades single-stranded DNA and RNA endonucleolytically to yield 5′-phosphoryl-terminated products. Double-stranded nucleic acids (DNA:DNA, DNA:RNA or RNA:RNA) are resistant to S1 nuclease degradation except with extremely high concentrations of enzyme. Lambda Exonuclease catalyzes the removal of 5′ mononucleotides from duplex DNA. Its preferred substrate is 5′-phosphorylated double stranded DNA, although Lambda Exonuclease will also degrade single-stranded and non-phosphorylated substrates at a greatly reduced rate. Lambda Exonuclease is unable to initiate DNA digestion at nicks or gaps, RecJ is a single-stranded DNA specific exonuclease that catalyzes the removal of deoxy-nucleotide monophosphates from DNA in the 5′ to 3′ direction. T7 exonuclease catalyzing the removal of 5′ mononucleotides from duplex DNA. T7 Exonuclease catalyzes nucleotide removal from the 5′ termini or at gaps and nicks of double-stranded DNA.

Restriction endonucleases are another example of nucleases that may be used in connection with the methods of the present invention. Non-limiting examples of restriction endonucleases and their recognition sequences are provided in Table 1.

TABLE 1

Recognition Sequences for Restriction
Endonucleases.

| | RECOGNITION | | RECOGNITION |
|---|---|---|---|
| ENZYME | SEQUENCE | ENZYME | SEQUENCE |
| AatII | GACGTC | Fnu4H I | GCNGC |
| Acc65 I | GGTACC | Fok I | GGATG |
| Acc I | GTMKAC | Fse I | GGCCGGCC |
| Aci I | CCGC | Fsp I | TGCGCA |
| Acl I | AACGTT | Hae II | RGCGCY |
| Afe I | AGCGCT | Hae II | GGCC |
| Afl II | CTTAAG | Hga I | GACGC |
| Afl III | ACRYGT | Hha I | GCGC |
| Age I | ACCGGT | Hinc II | GTYRAC |
| Ahd I | GACNNNNNGTC | Hind III | AAGCTT |
| Alu I | AGCT | Hinf I | GANTC |
| Alw I | GGATC | HinP1 I | GCGC |
| AlwN I | CAGNNNCTG | Hpa I | GTTAAC |
| Apa I | GGGCCC | Hpa II | CCGG |
| ApaL I | GTGCAC | Hph I | GGTGA |
| Apo I | RAATTY | Kas I | GGCGCC |
| Asc I | GGCGCGCC | Kpn I | GGTACC |
| Ase I | ATTAAT | Mbo I | GATC |
| Ava I | CYCGRG | Mbo II | GAAGA |
| Ava II | GGWCC | Mfe I | CAATTG |
| Avr II | CCTAGG | Mlu I | ACGCGT |
| Bae I | NACNNNNGTAPyGN | Mly I | GAGTCNNNNN |
| BamH I | GGATCC | Mnl I | CCTC |
| Ban I | GGYRCC | Msc I | TGGCCA |
| Ban II | GRGCYC | Mse I | TTAA |
| Bbs I | GAAGAC | Msl I | CAYNNNNRTG |
| Bbv I | GCAGC | MspA1 I | CMGCKG |
| BbvC I | CCTCAGC | Msp I | CCGG |
| Bcg I | CGANNNNNNTGC | Mwo I | GCNNNNNNNGC |
| BciV I | GTATCC | Nae I | GCCGGC |
| Bcl I | TGATCA | Nar I | GGCGCC |
| Bfa I | CTAG | Nci I | CCSGG |
| Bgl I | GCCNNNNNGGC | Nco I | CCATGG |
| Bgl II | AGATCT | Nde I | CATATG |
| Blp I | GCTNAGC | NgoMI V | GCCGGC |
| Bmr I | ACTGGG | Nhe I | GCTAGC |
| Bpm I | CTGGAG | Nla III | CATG |
| BsaA I | YACGTR | Nla IV | GGNNCC |
| BsaB I | GATNNNNATC | Not I | GCGGCCGC |
| BsaH I | GRCGYC | Nru I | TCGCGA |
| Bsa I | GGTCTC | Nsi I | ATGCAT |
| BsaJ I | CCNNGG | Nsp I | RCATGY |
| BsaW I | WCCGGW | Pac I | TTAATTAA |
| BseR I | GAGGAG | PaeR7 U | CTCGAG |
| Bsg I | GTGCAG | Pci I | ACATGT |
| BsiE I | CGRYCG | PflF I | GACNNNGTC |
| BsiHKA I | GWGCWC | PflM I | CCAANNNNNTGG |
| BsiW I | CGTACG | Ple I | GAGTC |
| Bsl I | CCNNNNNNNGG | Pme I | GTTTAAAC |
| BsmA I | GTCTC | Pml I | CACGTG |
| BsmB I | CGTCTC | PpuM I | RGGWCCY |
| BsmF I | GGGAC | PshA I | GACNNNNGTC |
| Bsm I | GAATGC | Psi I | TTATAA |
| BsoB I | CYCGRG | PspG I | CCWGG |
| Bsp1286 I | GDGCHC | PspOM I | GGGCCC |
| BspD I | ATCGAT | Pst I | CTGCAG |
| BspE I | TCCGGA | Pvu I | CGATCG |
| BspH I | TCATGA | Pvu II | CAGCTG |
| BspM I | ACCTGC | Rsa I | GTAC |
| BsrB I | CCGCTC | Rsr II | CGGWCCG |
| BsrD I | GCAATG | Sac I | GAGCTC |
| BsrF I | RCCGGY | Sac II | CCGCGG |
| BsrG I | TGTACA | Sal I | GTCGAC |
| Bsr I | ACTGG | Sap I | GCTCTTC |
| BssH II | GCGCGC | Sau3A I | GATC |
| BssK I | CCNGG | Sau96 I | GGNCC |
| Bst4C I | ACNGT | Sbf I | CCTGCAGG |
| BssS I | CACGAG | Sca I | AGTACT |
| BstAP I | GCANNNNNTGC | ScrF I | CCNGG |
| BstB I | TTCGAA | SexA I | ACCWGGT |
| BstE II | GGTNACC | SfaN I | GCATC |
| BstF5 I | GGATGNN | Sfc I | CTRYAG |
| BstN I | CCWGG | Sfi I | GGCCNNNNNGGCC |
| BstU I | CGCG | Sfo I | GGCGCC |
| BstX I | CCANNNNNNTGG | SgrA I | CRCCGGYG |
| BstY I | RGATCY | Sma I | CCCGGG |
| BstZ17 I | GTATAC | Sm1 I | CTYRAG |
| Bsu36 I | CCTNAGG | SnaB I | TACGTA |
| Btg I | CCPuPyGG | Spe I | ACTAGT |
| Btr I | CACGTG | Sph I | GCATGC |
| Cac8 I | GCNNGC | Ssp I | AATATT |
| Cla I | ATCGAT | Stu I | AGGCCT |
| Dde I | CTNAG | Sty I | CCWWGG |
| Dpn I | GATC | Swa I | ATTTAAAT |
| Dpn II | GATC | Taq I | TCGA |
| Dra I | TTTAAA | Tfi I | GAWTC |
| Dra III | CACNNNGTG | Tli I | CTCGAG |
| Drd I | GACNNNNNNGTC | Tse I | GCWGC |
| Eae I | YGGCCR | Tsp45 I | GTSAC |
| Eag I | CGGCCG | Tsp509 I | AATT |
| Ear I | CTCTTC | TspR I | CAGTG |
| Eci I | GGCGGA | Tth111 I | GACNNNGTC |
| EcoN I | CCTNNNNNAGG | Xba I | TCTAGA |
| EcoO109 I | RGGNCCY | Xcm I | CCANNNNNNNNNTGG |
| EcoR I | GAATTC | Xho I | CTCGAG |
| EcoR V | GATATC | Xma I | CCCGGG |
| Fau I | CCCGCNNNN | Xmn I | GAANNNNTTC |

Where R = A or G, K = G or T, S = G or C, Y = C or T, M = A or C, W = A or T, B = not A (C, G , or T), H = not G (A, C, or T), D = not C (A, G or T), V = not T (A, C or G), and N = any nucleotide.

Those of ordinary skill in the art will be able to select an appropriate nuclease depending on the characteristics of the nucleic acid sequence that is being transfected in to the cell. For example, where the nucleic acid sequence is an RNA, then a ribonuclease should be used. Where the nucleic acid sequence is a DNA, then a deoxyribonuclease should be used. If a restriction endonuclease is used to digest DNA, the choice of enzyme can be based on the cutting frequency of the enzyme and the number of recognition sequences in the transfected DNA. A combination of nucleases may be used to achieve the desired amount of nucleic acid degradation.

In one embodiment of the present invention, DNase I is used to improve viability and transgene expression in transfected cells. DNase I acts on single- and double-stranded DNA, chromatin, and RNA:DNA hybrids. Typical applications of DNase I in molecular biology include the degradation of DNA template in transcription reactions, removal of contaminating genomic DNA from RNA samples, DNase I footprinting, and nick translation. DNase I has been added to cell cultures for the purpose of viral DNA removal for bioprocessing applications (Kemppainen et al. 2004). In that study it was reported that it was reported that DNase treatment had no effect on cell viability. DNase I has also been added to electroporated DNA-sperm suspensions to evaluate whether the DNA was taken into the sperm cells or merely adhered to or incorporated in the plasma membrane (Gagne et al. 1991). Another study reported that DNase I treatment could increase survival in physically injured cells, presumably due to its actin-depolymerization properties (Miyake et al. 2001). Pulmozyme® is a prescription inhalation drug containing recombinant human DNase I used for the treatment of cystic fibrosis (Genetech, Inc.).

In some methods of the present invention, DNase I is used to increase viability and transgene expression in transfected cells. These methods comprise: transfecting a cell with a nucleic acid sequence; and contacting the transfected cell with DNase I following transfection in a manner effective to enhance viability of the transfected cell. The inventors have demonstrated that mixing transfected cells with DNase I after transfection improved cell viability, thus giving rise to better transfection efficiency. Furthermore, DNase I treatment allowed the cells to better tolerate high DNA concentrations and higher electrical currents, which may also result in better transfection efficiency. One theory is that some DNA molecules may be become trapped in the membrane bilayer during transfection, and consequently cells may take longer to recover after transfection or not recover at all. Thus, removal of DNA molecules from the membrane bilayer using one or more nucleases can promote cell survival.

### B. Cell Transfection Methods

Transfection is a procedure for temporarily and reversibly permeabilizing the cytoplasmic membrane to permit the transfer of DNA molecules into the target cells. Complete recovery of the cytpoplasmic membrane is important in achieving optimal cell viability and ultimately in achieving efficient transgene expression. The present invention provides methods that enhance cell recovery following transfection and thus, result in more efficient transfection and transgene expression.

Not wishing to be bound by theory, it that during transfection some DNA molecules may be become trapped in the membrane bilayer, and consequently cells may take longer to recover after transfection or not recover at all. Thus, removal of DNA molecules from the membrane bilayer promotes cell recovery. DNA molecules could become trapped in the membrane bilayer as a result of essentially any transfection method. Examples of such transfection methods include electroporation; microinjection (Harland and Weintraub, 1985; U.S. Pat. No. 5,789,215, incorporated herein by reference); calcium phosphate precipitation (Graham and Van Der Eb, 1973; Chen and Okayama, 1987; Rippe et al., 1990); DEAE-dextran followed by polyethylene glycol (Gopal, 1985); direct sonic loading (Fechheimer et al., 1987); liposome mediated transfection (Nicolau and Sene, 1982; Fraley et al., 1979; Nicolau et al., 1987; Wong et al., 1980; Kaneda et al., 1989; Kato et al., 1991); microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); and agitation with silicon carbide fibers (Kaeppler et al., 1990; U.S. Pat. Nos. 5,302,523 and 5,464,765, each incorporated herein by reference). It is contemplated that post-transfection nuclease treatment may be used to improve cell viability following any of these and other transfection methods.

In one embodiment, the invention provides a method for increasing viability of a cell after electroporation. Such a method may comprise: transfecting a cell with a nucleic acid sequence by electroporation; and contacting the cell with a nuclease after electroporation in a manner effective to enhance the viability of the electroporated cell. As used herein, “electroporation” refers to application of an electrical current or electrical field to a cell to facilitate entry of a nucleic acid molecule or other molecule into the cell. One of skill in the art would understand that any method and technique of electroporation is contemplated by the present invention. Methods and apparatuses for electroporation are described in, for example, U.S. patent application Ser. No. 10/225,446, filed Aug. 21, 2002; U.S. Pat. Nos. 5,612,207, 5,720,921 6,074,605, 6,090,617, 6,485,961, 6,773,669, 6,090,617, and 6,617,154; PCT Application Nos. WO 03/018751 and WO 2004/031353; and U.S. patent application Ser. Nos. 10/781,440, 10/080,272, and 10/675,592; all of which are incorporated by reference.

Electroporation has been described as a means to introduce nonpermeant molecules into living cells (reviewed in Mir, 2000). At the level of the entire cell, the consequences of cell exposure to the electric pulses are not completely understood. In the presence of the external electric field, a change in the transmembrane potential difference is believed to be generated (Neumann et al., 1999; Weaver and Chizmadzhev, 1996; Kakorin et al., 1996). It superimposes upon the resting transmembrane potential difference and it may be calculated from the Maxwell's equations, providing a few approximations are made (very reduced thickness of the cell membrane, null membrane conductivity, etc.) (Mir, 2000). These changes in the transmembrane potential difference have been experimentally observed (Hibino et al., 1993; Gabriel and Teissié, 1999). Analytically, the effects of the exposure of cells to electric pulses are well described in the case of isolated cells in suspension (Kotnik et al., 1998).

At the molecular level of analysis, the explanation of the phenomena occurring at the cell membrane level is hypothetical. It is assumed that above a threshold value of the net transmembrane potential, the changes occurring in membrane structure will be enough as to render that membrane permeable to otherwise nonpermeant molecules of given physicochemical characteristics (molecular mass, radius, etc.) (Mir, 2000).

DNA electroporation was originally described using simple generators that produce exponentially decaying pulses. Square-wave electric pulse generators were later developed that allowed specification of the various electric parameters (pulse intensity, pulse length, number of pulses) (Rols and Teissié, 1990). The selection of parameters is dependent on the cell type being electroporated and physical characteristics of the molecules that are to be taken up by the cell.

As generally practiced in vitro, electroporation is carried out in small (less than 0.5 milliliters) cuvette-like chambers containing a pair of electrodes with motionless cells and fluid (“static” EP). The volume of chambers for static EP determines the maximal amount of cells that can be conveniently electroporated. Static EP devices typically electroporate enough cells for many laboratory research applications but not enough for either industrial applications or cell-based therapy. Theoretically, large volumes could be electroporated by pooling large numbers of small batches from static electroporation. This, however, would be time consuming or require simultaneous use of multiple electroporation apparatuses, which would be costly and exacerbate problems of reproducibility and quality assurance.

Flow EP and streaming EP are two technologies that enable the processing of large volumes of cells. Thus, flow EP and streaming EP may be better suited to industrial applications and cell-based therapy than is static EP. Flow electroporation can be further divided into continuous flow electroporation and variable flow electroporation. In the continuous flow electroporation, a pulsed electric field (EF) is typically applied to cells continuously flowing through the chamber with the cells obtaining a desired number of electric pulse treatments. In variable flow electroporation, cells are typically processed in cycles. Each processing cycle typically involves: flowing cells into chamber in a controlled manner, resting cells for a definite time (>=0 min), electroporating cells in the chamber, and flowing cells out of chamber in a controlled way after a post-electroporation resting time (>=0 min). Variable flow EP is described in more detail in U.S. application Ser. No. 11/127,557, which is incorporated herein by reference. The movement of cells through a flow EP apparatus may be performed by computer-controlled electronic switches and/or pump(s). Of course, it is not required that the flow of cells through the apparatus be computer controlled.

In both static and conventional flow EP methods, the transient nature of the electric field experienced by the sample being electroporated may be the result of electronic control over the magnitude and duration of one or more voltage pulses applied to the electrodes. In the case of flow EP, the flow rate of cells between the electrodes is typically coordinated with the rate of high-voltage pulse application.

With “streaming” electroporation, a sample may be “pulsed” by its movement across electrical field lines. This, of course, is in contrast to techniques in which the duration of an electrical pulse (or pulses) applied to electrodes primarily determines the exposure of the sample to an electric field. In other words, in streaming EP, the rate of relative motion between an electric field and a sample can be used to achieve electroporation instead of signal pulsing applied to the electrodes. Streaming EP can utilize signal pulsing, although that pulsing no longer acts as the primary mechanism for achieving electroporation. Streaming electroporation is described in more detail in U.S. application Ser. No. 10/675,592, which is incorporated herein by reference.

Electroporation can mediate efficient gene delivery to cells. In practice, optimal transfection can be achieved by balancing the input electrical energy and the transfected cell viability. Electric pulses are a stress on the cell that can reduce viability. DNA molecules passing through the cell membrane bilayer via electroporation may also present an additional stress to the cell. Larger DNA molecules move slower in an electric field, which may partially explain why electroporating cells with large DNA molecules usually results in poor cell viability and transgene expression.

The present invention demonstrated that the post-EP digestion of nucleic acid sequences improved the viability of the transfected cells and enhanced transgene expression. The present invention also demonstrated that post-EP DNase I treatment allowed cells to tolerate higher energy electrical pulses and larger DNA molecules.

### C. Nucleic Acid-Based Expression Systems

1. Vectors

The present invention is useful for enhancing transgene expression in target cells. The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Goodboum and Maniatis et al., 1988 and Ausubel et al., 1996, both incorporated herein by reference).

The term “expression vector” refers to any type of genetic construct comprising a nucleic acid coding for an RNA capable of being transcribed and then translated into a protein, polypeptide, or peptide. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

a. Promoters and Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are commonly used in recombinant DNA construction include the β-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles, such as mitochondria, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 2001, incorporated herein by reference). The promoters employed may be constitutive, tissue-specific, inducible, and/or useful under the appropriate conditions to direct high-level expression of the introduced DNA segment, such as is advantageous in the large-scale production of recombinant proteins and/or peptides. The promoter may be heterologous or endogenous.

Additionally any promoter/enhancer combination (as per, for example, the Eukaryotic Promoter Data Base EPDB) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Table 2 lists non-limiting examples of elements/promoters that may be employed, in the context of the present invention, to regulate the expression of a RNA. Table 3 provides non-limiting examples of inducible elements, which are regions of a nucleic acid sequence that can be activated in response to a specific stimulus.

TABLE 2
Promoter and/or Enhancer

| Promoter/Enhancer | References |
|---|---|
| Immunoglobulin | Banerji et al., 1983; Gilles et al., |
| Heavy Chain | 1983; Grosschedl et al., 1985; |
| | Atchinson et al., 1986, 1987; |
| | Imler et al., 1987; Weinberger et al., |
| | 1984; Kiledjian et al., 1988; |
| | Porton et al.; 1990 |
| Immunoglobulin | Queen et al., 1983; Picard et al., 1984 |
| Light Chain | |
| T-Cell Receptor | Luria et al., 1987; Winoto et al., 1989; |
| | Redondo et al.; 1990 |
| HLA DQ a and/or | Sullivan et al., 1987 |
| DQ β | |
| β-Interferon | Goodbourn et al., 1986; Fujita et al., |
| | 1987; Goodbourn et al., 1988 |
| Interleukin-2 | Greene et al., 1989 |
| Interleukin-2 | Greene et al., 1989; Lin et al., 1990 |
| Receptor | |
| MHC Class II 5 | Koch et al., 1989 |
| MHC Class II | Sherman et al., 1989 |
| HLA-Dra | |
| β-Actin | Kawamoto et al., 1988; Ng et al.; 1989 |
| Muscle Creatine | Jaynes et al., 1988; Horlick et al., 1989; |
| Kinase (MCK) | Johnson et al., 1989 |
| Prealbumin | Costa et al., 1988 |
| (Transthyretin) | |
| Elastase I | Ornitz et al., 1987 |
| Metallothionein | Karin et al., 1987; Culotta et al., 1989 |
| (MTII) | |
| Collagenase | Pinkert et al., 1987; Angel et al., 1987 |
| Albumin | Pinkert et al., 1987; Tronche et al., 1989, |
| | 1990 |
| α-Fetoprotein | Godbout et al., 1988; Campere et al., 1989 |
| γ-Globin | Bodine et al., 1987; Perez-Stable et al., |
| | 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell | Hirsch et al., 1990 |
| Adhesion | |
| Molecule (NCAM) | |
| α₁-Antitrypsin | Latimer et al., 1990 |
| H2B (TH2B) Histone | Hwang et al., 1990 |
| Mouse and/or Type I | Ripe et al., 1989 |
| Collagen | |
| Glucose-Regulated | Chang et al., 1989 |
| Proteins (GRP94 | |
| and GRP78) | |
| Rat Growth Hormone | Larsen et al., 1986 |
| Human Serum Amyloid | Edbrooke et al., 1989 |
| A (SAA) | |
| Troponin I (TN I) | Yutzey et al., 1989 |
| Platelet-Derived | Pech et al., 1989 |
| Growth Factor (PDGF) | |
| Duchenne Muscular | Klamut et al., 1990 |
| Dystrophy | |
| SV40 | Banerji et al., 1981; Moreau et al., 1981; |
| | Sleigh et al., 1985; Firak et al., 1986; |
| | Herr et al., 1986; Imbra et al., 1986; |
| | Kadesch et al., 1986; Wang et al., 1986; |
| | Ondek et al., 1987; Kuhl et al., 1987; |
| | Schaffner et al., 1988 |
| Polyoma | Swartzendruber et al., 1975; Vasseur et al., |
| | 1980; Katinka et al., 1980, 1981; Tyndell et |
| | al., 1981; Dandolo et al., 1983; de Villiers |
| | et al., 1984; Hen et al., 1986; Satake et al., |
| | 1988; Campbell and/or Villarreal, 1988 |
| Retroviruses | Kriegler et al., 1982, 1983; Levinson et al., |
| | 1982; Kriegler et al., 1983, 1984a, b, 1988; |
| | Bosze et al., 1986; Miksicek et al., 1986; |
| | Celander et al., 1987; Thiesen et al., 1988; |
| | Celander et al., 1988; Choi et al., 1988; |
| | Reisman et al., 1989 |
| Papilloma Virus | Campo et al., 1983; Lusky et al., 1983; |
| | Spandidos and/or Wilkie, 1983; Spalholz et al., |
| | 1985; Lusky et al., 1986; Cripe et al., 1987; |
| | Gloss et al., 1987; Hirochika et al., 1987; |
| | Stephens et al., 1987 |
| Hepatitis B Virus | Bulla et al., 1986; Jameel et al., 1986; |
| | Shaul et al., 1987; Spandau et al., 1988; |
| | Vannice et al., 1988 |
| Human Immuno- | Muesing et al., 1987; Hauber et al., 1988; |
| deficiency Virus | Jakobovits et al., 1988; Feng et al., 1988; |
| | Takebe et al., 1988; Rosen et al., 1988; |
| | Berkhout et al., 1989; Laspia et al., 1989; |
| | Sharp et al., 1989; Braddock et al., 1989 |
| Cytomegalovirus | Weber et al., 1984; Boshart et al., 1985; |
| (CMV) | Foecking et al., 1986 |
| Gibbon Ape Leukemia | Holbrook et al., 1987; Quinn et al., 1989 |
| Virus | |
| CMV/α-Act | Okabe et al., 1997 |
| α-Act/RU5′ | Takebe et al., 1988 |
| EF1α/RU5′ | Kim et al., 1990; Guo et al., 1996 |
| CMV-hFerL-chEF1α | Eisenstein and Munro, 1990; Boshart et al., |
| | 1985 |
| SV40- hFerL-chEF1α | Eisenstein and Munro, 1990; Moreau, 1981 |
| PGK (phospho- | Singer-Sam et al., 1984 |
| glycerate kinase) | |
| Ubiquitinase | Christensen et al., 1996 |
| CMV-Ub | Yew, 2001 |

TABLE 3
Inducible Elements

| Element | Inducer | References |
|---|---|---|
| MT II | Phorbol Ester | Palmiter et al., 1982; |
| | (TFA) Heavy metals | Haslinger et al., 1985; |
| | | Searle et al., 1985; |
| | | Stuart et al., 1985; |
| | | Imagawa et al., 1987, |
| | | Karin et al., 1987; |
| | | Angel et al., 1987b; |
| | | McNeall et al., 1989 |
| MMTV (mouse | Glucocorticoids | Huang et al., 1981; |
| mammary tumor | | Lee et al., 1981; |
| virus) | | Majors et al., 1983; |
| | | Chandler et al., 1983; |
| | | Lee et al., 1984; |
| | | Ponta et al., 1985; |
| | | Sakai et al., 1988 |
| β-Interferon | Poly(rI)x | Tavernier et al., 1983 |
| | Poly(rc) | |
| Adenovirus 5 E2 | ElA | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene | Interferon | Blanar et al., 1989 |
| H-2κb | | |
| HSP70 | ElA, SV40 Large T | Taylor et al., 1989, |
| | Antigen | 1990a, 1990b |
| Proliferin | Phorbol Ester-TPA | Mordacq et al., 1989 |
| Tumor Necrosis | PMA | Hensel et al., 1989 |
| Factor α | | |
| Thyroid Stimula- | Thyroid Hormone | Chatterjee et al., |
| ting Hormone α | | 1989 |
| Gene | | |

The identity of tissue-specific promoters or elements, as well as assays to characterize their activity, is well known to those of skill in the art. Non-limiting examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), DIA dopamine receptor gene (Lee, et al., 1997), insulin-like growth factor II (Wu et al., 1997), and human platelet endothelial cell adhesion molecule-1 (Almendro et al., 1996).

b. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

The use of internal ribosome entry sites (IRES) elements may be used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

c. Multiple Cloning Sites

Vectors can include a multiple cloning site (MCS), which is a nucleic acid region that contains multiple restriction enzyme sites, any of which can be used in conjunction with standard recombinant technology to digest the vector (see, for example, Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

d. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, Chandler et al., 1997, herein incorporated by reference.)

e. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to be more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

f. Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal or the bovine growth hormone polyadenylation signal, convenient and known to function well in various target cells. Polyadenylation may increase the stability of the transcript or may facilitate cytoplasmic transport.

g. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

h. Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is calorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

i. Plasmid Vectors

In certain embodiments, a plasmid vector is contemplated for use to transform a cell. In general, plasmid vectors containing replicon and control sequences which are derived from species compatible with the cell are used in connection with these cells. The vector ordinarily carries a replication site, as well as marking sequences which are capable of providing phenotypic selection in transformed cells.

### D. Host Cells and Expression Systems

1. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organisms that is capable of replicating a vector and/or expressing a heterologous gene encoded by a vector. A host cell may be “transfected” or “transformed,” which refers to a process by which exogenous nucleic acid, such as a modified protein-encoding sequence, is transferred or introduced into the host cell. A transformed cell includes the primary subject cell and its progeny.

Host cells may be derived from prokaryotes or eukaryotes, including bacteria cells, insect cells, plant cells, and mammalian cells, depending upon whether the desired result is replication of the vector or expression of part or all of the vector-encoded nucleic acid sequences. Host cells may be primary cells or established cell lines. Numerous cell lines and cultures are available for use as a host cell, and they can be obtained through the American Type Culture Collection (ATCC), which is an organization that serves as an archive for living cultures and genetic materials. An appropriate host can be determined by one of skill in the art based on the vector backbone and the desired result. Examples of mammalian cells that can be used in the context of the present invention include, but are not limited to, human embryonic kidney cells, K562 cells, Jurkat cells, 293T cells, Vero cells, CHO cells, HeLa cells, W138, BHK, COS-7, HepG2, 3T3, RIN and MDCK cells or any eukaryotic cells for which tissue culture techniques are established.

In certain embodiments, it may be useful to employ selection systems that preclude growth of undesirable cells. This may be accomplished by virtue of permanently transforming a cell line with a selectable marker or by transducing or infecting a cell line with a vector that encodes a selectable marker. In either situation, culture of the transformed/transduced cell with an appropriate drug or selective compound will result in the enhancement, in the cell population, of those cells carrying the marker.

Examples of markers include, but are not limited to, HSV thymidine kinase, hypoxanthine-guanine phosphoribosyltransferase and adenine phosphoribosyltransferase genes, in tk-, hgprt- or aprt-cells, respectively. Also, anti-metabolite resistance can be used as the basis of selection for dhfr, that confers resistance to methotrexate; gpt, that confers resistance to mycophenolic acid; neo, that confers resistance to the aminoglycoside G418; and hygro, that confers resistance to hygromycin.

2. Cell Culture Systems

In eukaryotic cell culture systems, the culture of the cells is generally under conditions of controlled pH, temperature, humidity, osmolarity, ion concentrations, and exchange of gases. Regarding the latter, oxygen and carbon dioxide are of particular importance to the culturing of cells. In a typical eukaryotic cell culture system, an incubator is provided in which carbon dioxide is infused to maintain an atmosphere of about 5% carbon dioxide within the incubator. The carbon dioxide interacts with the tissue culture medium, particularly its buffering system, in maintaining the pH near physiologic levels.

In addition to carbon dioxide, the culturing of cells is dependent upon the ability to supply to the cells a sufficient amount of oxygen necessary for cell respiration and metabolic function. Methods to increase oxygen concentration to the cultured cells include mechanical stirring, medium perfusion or aeration, increasing the partial pressure of oxygen, and/or increasing the atmospheric pressure.

Conventional cell culture containers comprise tissue culture flasks, tissue culture bottles, and tissue culture plates. Gas exchange between the incubator atmosphere and a tissue culture plate generally involves a loosely fitting cover which overhangs the plate. Similarly, for a tissue culture flasks or bottle, a loosely fitting cap excludes particulate contaminants from entering the chamber of the flask or bottle, but allows gas exchange between the incubator atmosphere and the atmosphere within the flask or bottle. Caps with a gas permeable membrane or filter are also available, thereby allowing for gas exchange with a tightly fitting cap.

As used herein, “media” and “medium” refers to any substance which can facilitate growth of cells. One of skill in the art would be familiar with the wide range of types of media available which can be used in cell culture systems. In certain embodiments of the present invention, the host cells are grown in media that is serum-free media. In other embodiments of the present invention, the host cells are grown in media that is protein-free media. One of skill in the art would understand that various components and agents can be added to the media to facilitate and control cell growth. For example, the glucose concentration of the media can be maintained at a certain level.

Mammalian cells can be propagated in vitro in two modes: as non-anchorage dependent cells growing freely in suspension throughout the bulk of the culture; or as anchorage-dependent cells requiring attachment to a solid substrate for their propagation (i.e., a monolayer type of cell growth). Traditionally, anchorage-dependent cell cultures are propagated on the bottom of small glass or plastic vessels. A number of techniques have been proposed that offer large accessible surfaces for cell growth: the roller bottle system, the stack plates propagator, the spiral film bottle, the hollow fiber system, the packed bed, the plate exchanger system, and the membrane tubing reel. The roller bottle system is a commonly used process for large scale anchorage-dependent cell production. Fully automated robots are available that can handle thousands of roller bottles per day, thus eliminating the risk of contamination and inconsistency associated with the otherwise required intense human handling. With frequent media changes, roller bottle cultures can achieve cell densities of close to 0.5×10⁶ cells/cm² (corresponding to approximately 10⁹ cells/bottle or almost 10⁷ cells/ml of culture media).

In an effort to overcome the shortcomings of the traditional anchorage-dependent culture processes, van Wezel (1967) developed the concept of microcarrier culturing systems. In this system, cells are propagated on the surface of small solid particles suspended in the growth medium by slow agitation. Cells attach to the microcarriers and grow gradually to confluency on the microcarrier surface. In fact, this large scale culture system upgrades the attachment dependent culture from a single disc process to a unit process in which both monolayer and suspension culture have been brought together. Thus, combining the necessary surface for a cell to grow with the advantages of the homogeneous suspension culture increases production.

The advantages of microcarrier cultures over most other anchorage-dependent, large-scale cultivation methods are several fold. First, microcarrier cultures offer a high surface-to-volume ratio (variable by changing the carrier concentration), which leads to high cell density yields and a potential for obtaining highly concentrated cell products. Cell yields are up to 1-2×10⁷ cells/ml when cultures are propagated in a perfused reactor mode. Second, cells can be propagated in one unit process vessels instead of using many small low-productivity vessels (i.e., flasks or dishes). Third, the well-mixed and homogeneous microcarrier suspension culture makes it possible to monitor and control environmental conditions (e.g., pH, pO₂, and concentration of medium components), thus leading to more reproducible cell propagation and product recovery. Fourth, it is possible to take a representative sample for microscopic observation, chemical testing, or enumeration. Fifth, since microcarriers settle out of suspension quickly, use of a fed-batch process or harvesting of cells can be done relatively easily. Sixth, the mode of the anchorage-dependent culture propagation on the microcarriers makes it possible to use this system for other cellular manipulations, such as: cell transfer without the use of proteolytic enzymes; cocultivation of cells; transplantation into animals; and perfusion of the culture using decanters, columns, fluidized beds, or hollow fibers for microcarrier retainment. Seventh, microcarrier cultures are relatively easily scaled up using conventional equipment used for cultivation of microbial and animal cells in suspension.

One method which has shown to be particularly useful for culturing mammalian cells is microencapsulation. The mammalian cells are retained inside a semipermeable hydrogel membrane. A porous membrane is formed around the cells permitting the exchange of nutrients, gases, and metabolic products with the bulk medium surrounding the capsule. Several methods have been developed that are gentle, rapid and non-toxic and where the resulting membrane is sufficiently porous and strong to sustain the growing cell mass throughout the term of the culture. These methods are all based on soluble alginate gelled by droplet contact with a calcium-containing solution. Lim (1982, U.S. Pat. No. 4,352,883, incorporated herein by reference), describes cells concentrated in an approximately 1% solution of sodium alginate that are forced through a small orifice, forming droplets, and breaking free into an approximately 1% calcium chloride solution. The droplets are then cast in a layer of polyamino acid that ionically bonds to the surface alginate. Finally the alginate is reliquified by treating the droplet in a chelating agent to remove the calcium ions. Other methods use cells in a calcium solution to be dropped into an alginate solution, thus creating a hollow alginate sphere. A similar approach involves cells in a chitosan solution dropped into alginate, also creating hollow spheres.

Microencapsulated cells are easily propagated in stirred tank reactors and, with bead sizes in the range of 150-1500 μm in diameter, are easily retained in a perfused reactor using a fine-meshed screen. The ratio of capsule volume to total media volume can be maintained from as dense as 1:2 to 1:10. With intracapsular cell densities of up to 10⁸, the effective cell density in the culture is 1-5×10⁷. The advantages of microencapsulation include: the protection from the deleterious effects of shear stresses that occur from sparging and agitation, the ability to easily retain beads for the purpose of using perfused systems, the ability to scale up the process, and the ability to use the beads for implantation.

Perfusion refers to continuous flow at a steady rate, through or over a population of cells of a physiological nutrient solution. It implies the retention of the cells within the culture unit as opposed to continuous-flow culture, which washes the cells out with the withdrawn media (e.g., chemostat). The technique was initiated to mimic the cells milieu in vivo where cells are continuously supplied with blood, lymph, or other body fluids. Without perfusion, cells in culture go through alternating phases of being fed and starved, thus limiting full expression of their growth and metabolic potential.

The current use of perfused culture is in response to the challenge of growing cells at high densities (e.g., 0.1-5×10⁸ cells/ml). In order to increase densities beyond 2-4×10⁶ cells/ml, the medium has to be constantly replaced with a fresh supply in order to make up for nutritional deficiencies and to remove toxic products. Perfusion allows for a far better control of the culture environment (pH, pO₂, nutrient levels, etc.) and is a means of significantly increasing the utilization of the surface area within a culture for cell attachment.

The development of a perfused packed-bed reactor using a bed matrix of a non-woven fabric has provided a means for maintaining a perfusion culture at densities exceeding 10⁸ cells/ml of the bed volume (CelliGen™, New Brunswick Scientific, Edison, N.J.; Wang et al., 1992; Wang et al., 1993; Wang et al., 1994). Briefly described, this reactor comprises an improved reactor for culturing of both anchorage- and non-anchorage-dependent cells. The reactor is designed as a packed bed with a means to provide internal recirculation. Preferably, a fiber matrix carrier is placed in a basket within the reactor vessel. A top and bottom portion of the basket has holes, allowing the medium to flow through the basket. A specially designed impeller provides recirculation of the medium through the space occupied by the fiber matrix for assuring a uniform supply of nutrient and the removal of wastes. This simultaneously assures that a negligible amount of the total cell mass is suspended in the medium. The combination of the basket and the recirculation also provides a bubble-free flow of oxygenated medium through the fiber matrix. The fiber matrix is a non-woven fabric having a “pore” diameter of from 10 μm to 100 μm, providing for a high internal volume with pore volumes corresponding to 1 to 20 times the volumes of individual cells.

The perfused packed-bed reactor offers several advantages. With a fiber matrix carrier, the cells are protected against mechanical stress from agitation and foaming. The free medium flow through the basket provides the cells with optimum regulated levels of oxygen, pH, and nutrients. Products can be continuously removed from the culture and the harvested products are free of cells and can be produced in low-protein medium, which facilitates subsequent purification steps. Also, the design of this reactor system makes it possible to scale up the reactor. This technology is explained in detail in WO 94/17178 (Aug. 4, 1994, Freedman et al), which is hereby incorporated by reference in its entirety.

The Cellcube™ (Corning-Costar) module provides a large styrenic surface area for the immobilization and growth of substrate attached cells. It is an integrally encapsulated sterile single-use device that has a series of parallel culture plates joined to create thin sealed laminar flow spaces between adjacent plates.

The Cellcube™ module has inlet and outlet ports that are diagonally opposite each other and help regulate the flow of media. During the first few days of growth the culture is generally satisfied by the media contained within the system after initial seeding. The amount of time between the initial seeding and the start of the media perfusion is dependent on the density of cells in the seeding inoculum and the cell growth rate. The measurement of nutrient concentration in the circulating media is a good indicator of the status of the culture. When establishing a procedure it may be necessary to monitor the nutrients composition at a variety of different perfusion rates to determine the most economical and productive operating parameters.

Cells within the system reach a higher density of solution (cells/ml) than in traditional culture systems. Many typically used basal media are designed to support 1-2×10⁶ cells/ml/day. A typical Cellcube™, run with an 85,000 cm² surface, contains approximately 6 L media within the module. The cell density often exceeds 10⁷ cells/mL in the culture vessel. At confluence, 2-4 reactor volumes of media are required per day.

The timing and parameters of the production phase of cultures depends on the type and use of a particular cell line. Many cultures require a different media for production than is required for the growth phase of the culture. The transition from one phase to the other will likely require multiple washing steps in traditional cultures. However, the Cellcube™ system employs a perfusion system. One of the benefits of such a system is the ability to provide a gentle transition between various operating phases. The perfusion system negates the need for traditional wash steps that seek to remove serum components in a growth medium.

Suspension culture systems are particularly suitable for use in the present invention, as they reduce the amount of handling required to electroporate and culture the cells. For example, cells growing in a bioreactor can be transferred to an electroporation chamber for transfection and then to a bioreactor for further culture. The movement of the cells through the system may be automated. Furthermore, coupling the cell culture system to a flow electroporation system or a streaming electroporation system would allow rapid, large-scale processing.

Two suspension culture bioreactor designs are widely used in the industry due to their simplicity and robustness of operation—the stirred bioreactor and the airlift bioreactor. Agitation of the culture medium may also be achieved by axial rocking of a planar platform to induce wave motions inside of the bioreactor. The stirred bioreactor design has successfully been used on a scale of 8000 liter capacity for the production of interferon (Phillips et al., 1985; Mizrahi, 1983). Cells are grown in a stainless steel tank with a height-to-diameter ratio of 1:1 to 3:1. The culture is usually mixed with one or more agitators, based on bladed disks or marine propeller patterns. Agitator systems offering less shear forces than blades have been described. Agitation may be driven either directly or indirectly by magnetically coupled drives. Indirect drives reduce the risk of microbial contamination through seals on stirrer shafts.

The airlift bioreactor, also initially described for microbial fermentation and later adapted for mammalian culture, relies on a gas stream to both mix and oxygenate the culture. The gas stream enters a riser section of the bioreactor and drives circulation. Gas disengages at the culture surface, causing denser liquid free of gas bubbles to travel downward in the downcomer section of the bioreactor. The main advantage of this design is the simplicity and lack of need for mechanical mixing. Typically, the height-to-diameter ratio is 10:1. The airlift reactor scales up relatively easily, has good mass transfer of gasses and generates relatively low shear forces.

Most large-scale suspension cultures are operated as batch or fed-batch processes because they are the most straightforward to operate and scale up. However, continuous processes based on chemostat or perfusion principles are available.

A batch process is a closed system in which a typical growth profile is seen. A lag phase is followed by exponential, stationary and decline phases. In such a system, the environment is continuously changing as nutrients are depleted and metabolites accumulate. This makes analysis of factors influencing cell growth and productivity, and hence optimization of the process, a complex task. Productivity of a batch process may be increased by controlled feeding of key nutrients to prolong the growth cycle. Such a fed-batch process is still a closed system because cells, products, and waste products are not removed.

In what is still a closed system, perfusion of fresh medium through the culture can be achieved by retaining the cells with a variety of devices (e.g. fine mesh spin filter, hollow fiber or flat plate membrane filters, settling tubes). A true open system and the simplest perfusion process is the chemostat in which there is an inflow of medium and an outflow of cells and products. Culture medium is fed to the reactor at a predetermined and constant rate which maintains the dilution rate of the culture at a value less than the maximum specific growth rate of the cells (to prevent washout of the cell mass from the reactor). Culture fluid containing cells and cell products and byproducts is removed at the same rate. One of skill in the art would be familiar with the various types of filters that can be used for perfusion of media, and the various methods that can be employed for attaching the filter to the bioreactor and incorporating it into the cell growth process.

### E. Protein, Virus, and Transgenic Cell Production

As mentioned above, the present invention is directed to methods of improving the viability of transfected cells and increasing transgene expression. The methods described herein provide increased numbers of viable cells and enhance transgene expression. Furthermore, the methods are readily compatible with large-volume cell culture systems and high-throughput electroporation systems. Consequently, the present invention is well suited for the large-scale production of proteins, viruses, and transgenic cells.

Therapeutic proteins, as well as proteins having other research, commercial, or industrial applicability, may be produced according to the methods of the present invention. In some aspects, these proteins may be purified for use in pharmaceutical preparations. In other aspects, the transgenic cells themselves may be used therapeutically. For example, autologous cancer cells modified according to the methods of the present invention to express one or more immunostimulatory proteins may be reintroduced into the patient as a cancer vaccine. As another example, antigen presenting cells may be transfected according to the methods of the present invention to express one or more antigens and then introduced into a patient. The present invention could also be used for the large-scale production of viral vectors by transient co-transfection of cells.

1. Therapeutic Proteins

The transfected cells of the present invention are modified to express one or more therapeutic proteins. A “therapeutic protein” is a protein that can be administered to a subject for the purpose of treating or preventing a disease. Examples of classes of therapeutic proteins include tumor suppressors, inducers of apoptosis, cell cycle regulators, immuno-stimulatory proteins, toxins, cytokines, enzymes, antibodies, inhibitors of angiogenesis, metalloproteinase inhibitors, hormones or peptide hormones.

a. Immuno-Stimulatory Proteins

In some embodiments of the invention, the therapeutic protein is an immuno-stimulatory protein. An “immuno-stimulatory protein” is a protein involved in the activation, differentiation, or chemotaxis of immune cells. Examples of classes of immuno-stimulatory proteins include cytokines and thymic hormones. Thymic hormones include, for example, prothymosin-α, thymulin, thymic humoral factor (THF), THF-γ-2, thymocyte growth peptide (TGP), thymopoietin (TPO), thymopentin, and thymosin-α-1.

The term cytokine refers to a diverse group of secreted, soluble proteins and peptides that mediate communication among cells and modulate the functional activities of individual cells and tissues. Classes of cytokines include interleukins, interferons, colony stimulating factors, and chemokines. Examples of cytokines include: IL-1α, IL-1β, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, leukocyte inhibitory factor (LIF), IFN-α, IFN-γ, TNF, TNF-α, TGF-β, G-CSF, M-CSF, and GM-CSF.

Interleukins are involved in processes of cell activation, cell differentiation, proliferation, and cell-to-cell interactions. Those of skill in the art are familiar with interleukins including, but not limited to: IL-1, IL-1α, IL-1β, IL-2, IL-3, IL-4, IL-5, IL-6, IL-7, IL-8, IL-9, IL-10, IL-11, IL-12, IL-13, IL-14, IL-15, IL-16, IL-17, IL-17B, IL-17C, IL-17E, IL-17F, IL-18, IL-19, IL-20, IL-21, IL-21, IL-22, IL-23, IL-24, IL-25, IL-26, IL-27, IL-28A, IL-28B, IL-29, and IL-30.

Interferons are proteins that possess antiviral, antiproliferative, and immunomodulating activities. In addition, interferons influence metabolism, growth, and differentiation of cells. IFN-α, IFN-β, and IFN-γ are the three main human interferons. IFN-γ, which is produced primarily by the Th1 type of lymphocytes, exhibits many immunoregulatory effects, including the ability to induce the differentiation and activation of T cells and macrophages. Colony stimulating factors include, for example, G-CSF, M-CSF, GM-CSF, IL-3, and MEG-CSA.

Chemokines are a family of pro-inflammatory activation-inducible cytokines, which are mainly chemotactic for different cell types. There are four major classes of chemokines: C-chemokines, CC-chemokines, CXC-chemokines, and CX3C-chemokines. Non-limiting examples of chemokines include MCP-1, MCP-2, MCP-3, MIP-1α/β, IP-10, MIG, IL-8, RANTES, and lymphotactin. Other immuno-stimulatory proteins that may be used in the methods and compositions of the present invention include B7.1 (CD80), B7.2 (CD86), CD40, CD40 Ligand (CD40L), LFA-1, ICAM-1, VLA-4, and VCAM-1.

b. Developmental Proteins

Developmental proteins is another class of proteins whose expression may be enhanced by using the compositions and methods of the present invention. Developmental genes include, for example, adhesion molecules, cyclin kinase inhibitors, Wnt family members, Pax family members, Winged helix family members, Hox family members, cytokines/lymphokines and their receptors, growth or differentiation factors and their receptors, neurotransmitters and their receptors.

c. Oncogenes and Tumor Suppressors

The methods of the present invention can be used to produce oncogenes and tumor suppressors. Non-limiting examples of oncogenes include ABLI, BLC1, BCL6, CBFA1, CBL, CSFIR, ERBA, ERBB, EBRB2, ETS1, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TAL1, TCL3, and YES. Non-limiting examples of tumor suppressor genes include p53, Rb, Rap1A, DCC, k-rev, BRCA1, BRCA2, zac1, p73, MMAC-1, ATM, HIC-1, DPC-4, FHIT, APC, DCC, PTEN, ING1, NOEY1, NOEY2, PML, OVCA1, MADR2, WT1, 53BP2, IRF-1, MADH4, MCC, NF1, NF2, RB1, TP53, and WT1.

d. Enzymes

Particularly appropriate genes for expression include enzyme-encoding genes. Enzymes are used for a wide-variety of therapeutic, research, commercial, and industrial purposes. Examples of useful gene products include carbamoyl synthetase I, ornithine transcarbamylase, arginosuccinate synthetase, arginosuccinate lyase, and arginase. Other desirable gene products include fumarylacetoacetate hydrolase, phenylalanine hydroxylase, alpha-1 antitrypsin, glucose-6-phosphatase, low-density-lipoprotein receptor, porphobilinogen deaminase, factor VIII, factor IX, cystathione β-synthase, branched chain ketoacid decarboxylase, albumin, isovaleryl-CoA dehydrogenase, propionyl CoA carboxylase, methyl malonyl CoA mutase, glutaryl CoA dehydrogenase, insulin, β-glucosidase, pyruvate carboxylase, hepatic phosphorylase, phosphorylase kinase, glycine decarboxylase (also referred to as P-protein), H-protein, T-protein, Menkes disease copper-transporting ATPase, and Wilson's disease copper-transporting ATPase.

Other examples include cytosine deaminase, hypoxanthine-guanine phosphoribosyltransferase, galactose-1-phosphate uridylyltransferase, galactokinase, UDP-galactose-4-epimerase, phenylalanine hydroxylase, glucocerbrosidase, sphingomyelinase, α-L-iduronidase, glucose-6-phosphate dehydrogenase, HSV thymidine kinase, and human thymidine kinase,

Other types of enzymes include ACP desaturases and hycroxylases, ADP-glucose pyrophorylases, ATPases, alcohol dehydrogenases, amylases, amyloglucosidases, catalases, cellulases, cyclooxygenases, decarboxylases, dextrinases, esterases, DNA and RNA polymerases, hyaluron synthases, galactosidases, glucanases, glucose oxidases, GTPases, helicases, hemicellulases, hyaluronidases, integrases, invertases, isomersases, kinases, lactases, lipases, lipoxygenases, lyases, lysozymes, pectinesterases, peroxidases, phosphatases, phospholipases, phophorylases, polygalacturonases, proteinases and peptideases, pullanases, recombinases, reverse transcriptases, topoisomerases, and xylanases.

e. Hormones

Hormones are another group of genes that may be produced according to the methods described herein. Included are growth hormone, prolactin, placental lactogen, luteinizing hormone, follicle-stimulating hormone, chorionic gonadotropin, thyroid-stimulating hormone, leptin, adrenocorticotropin (ACTH), angiotensin I and II, β-endorphin, β-melanocyte stimulating hormone (β-MSH), cholecystokinin, endothelin I, galanin, gastric inhibitory peptide (GIP), glucagon, insulin, lipotropins, neurophysins, somatostatin, calcitonin, calcitonin gene related peptide (CGRP), β-calcitonin gene related peptide, hypercalcemia of malignancy factor, parathyroid hormone-related protein (PTH-rP), glucagon-like peptide (GLP-1), pancreastatin, pancreatic peptide, peptide YY, PHM, secretin, vasoactive intestinal peptide (VIP), oxytocin, vasopressin (AVP), vasotocin, enkephalinamide, metorphinamide, alpha melanocyte stimulating hormone (alpha-MSH), atrial natriuretic factor (ANF), amylin, amyloid P component (SAP-1), corticotropin releasing hormone (CRH), growth hormone releasing factor (GHRH), luteinizing hormone-releasing hormone (LHRH), neuropeptide Y, substance K (neurokinin A), substance P, and thyrotropin releasing hormone (TRH).

f. Antigens

A therapeutic protein can also be an antigenic peptide or polypeptide capable of generating an immune response. Examples include polynucleotides encoding antigens such as viral antigens, bacterial antigens, fungal antigens or parasitic antigens. Virus targets include picornavirus, coronavirus, togavirus, flavivirus, rhabdovirus, paramyxovirus, orthomyxovirus, bunyavirus, arenvirus, reovirus, retrovirus, papovavirus, parvovirus, herpesvirus, poxvirus, hepadnavirus, and spongiform virus. Parasite targets include trypanosomes, tapeworms, roundworms, and helminthes. Also, tumor markers, such as fetal antigen or prostate specific antigen, may be targeted in this manner.

g. Other Proteins

Other examples of proteins that can be produced according to the methods of the present invention include blood derivatives; growth factors; neurotransmitters or their precursors or synthetic enzymes; trophic factors (such as BDNF, CNTF, NGF, IGF, GMF, aFGF, bFGF, NT3, NT5, and the like); apolipoproteins (such as ApoAI, ApoAIV, ApoE, and the like); dystrophin or a minidystrophin; genes coding for factors involved in coagulation (such as factors VII, VIII, IX, and the like); cytosine deaminase, or all or part of a natural or artificial immunoglobulin (Fab, ScFv, and the like); anti-thrombotic genes (e.g., COX-1, TFPI); genes involved in angiogenesis (e.g., VEGF, FGF, thrombospondin, BAI-1, GDAIF, or their receptors); MCC, and mouse or humanized monoclonal antibodies.

2. Protein Purification

It may be desirable to purify the proteins produced according to the present invention. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography, polyacrylamide gel electrophoresis, and isoelectric focusing. A particularly efficient method of purifying peptides is fast protein liquid chromatography or HPLC.

Certain aspects of the present invention concern the purification, and in particular embodiments, the substantial purification, of an encoded protein or peptide. The purified proteins can be used in pharmaceutical compositions or for research, commercial, or industrial applications. The term “purified protein or peptide” as used herein, is intended to refer to a composition, isolatable from other components, wherein the protein or peptide is purified to any degree from the components of the cell in which it was produced. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

3. Transgenic Cells for Cell Therapy

The present invention can also be used to produce more efficiently transgenic cells for use in cell therapy. With cell-based therapy, cells are genetically modified ex vivo, and then reintroduced into the subject. The methods disclosed herein can be used to increase the number of cells that survive this ex vivo manipulation.

There are a variety of cell therapy approaches known in the art. Therapies involving secreted proteins are amenable to treatment using this approach. Exemplary secreted proteins include cytokines, colony stimulating factors, nerve growth factors, and hormones.

The methods described herein could be used to more efficiently produce genetically modified tumor cells. The tumor cells could be modified to overexpress one or more immuno-stimulatory proteins. Once transfected with the immuno-stimulatory protein(s), the cells could be irradiated, or otherwise inactivated, and administered to a cancer patient, in order to stimulate an immune response against the tumor cells. Methods and compositions relating to cancer vaccines are disclosed in the U.S. Provisional Patent Application entitled “Genetically Modified Tumor Cells as Cancer Vaccines” by Liu et al., filed Dec. 10, 2004, incorporated herein by reference.

The methods described herein could also be used to enhance the expression of antigens from antigen presenting cells. For example, antigen presenting cells could be loaded with nucleic acid vectors encoding one or more antigens ex vivo according the methods of the present invention. The transfected cells could then be administered to a patient in order to stimulate an immune response.

4. Viral Vector Production

The methods of the present invention are useful in the production of viral vectors. Viruses are highly efficient at nucleic acid delivery to specific cell types, while often avoiding detection by the infected host's immune system. These features make certain viruses attractive candidates as gene-delivery vehicles for use in gene therapies (Robbins and Ghivizzani, 1998; Cristiano et al., 1998).

Current transient transfection methods, such as CaPO₄ and small static electroporation, allow production of small volumes of viral vectors at a time, and the process is cumbersome, expensive and inefficient. Methods employing CaPO₄ can introduce inconsistencies from lot to lot, which can potentially lead to regulatory issues. Furthermore, precipitation of CaPO₄ interferes with downstream purification/concentration of viral particles, which withholds some human clinical trials because a 1000-fold virus concentration is typically needed.

Scaling up of production for replication-incompetent viral vectors is a major hurdle for large gene therapy clinical trials. Transient, simultaneous transfection of cells with multiple plasmids results in the production of vectors and decreases the possibility of viral-genome recombination. The methods of the present invention are capable of producing cells that yield high virus titers, and thus provides a safe and reliable method, which can be used for large-scale viral vector production, including retrovirus, lentivirus, adenovirus, AAV, and alphavirus vector productions. The production of infectious vectors by electroporation-mediated co-transfection of cells is described in U.S. patent application Ser. No. 10/751,586, incorporated herein by reference.

5. Other Nucleic Acids

The methods of the present invention are useful in the delivery of nucleic acid sequences that are useful in themselves or in the delivery of expression vectors that encode such nucleic acid sequences. Nucleic acid sequences that are not transcribed or translated include, for example, aptamers, ribosomal RNA, tRNA, splicosomal RNA, antisense RNA, siRNA, and mRNA.

### F. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### DNase Treatment Improves Cell Viability and Transfection Efficiency in Jurkat Cells by Allowing the Cells to Tolerate Higher Input Electrical Energy

A DNase stock solution was prepared by reconstituting the lyophilized DNase in electroporation (EP) buffer at a concentration of 2000 U/mL. The DNase was added to the transfected cells in a ratio of 1 volume DNase stock solution to 1 volumes of transfected cells.

Jurkat cells were transfected with either the plasmid pTM2 (pCMV-eGFP on pCI backbone (Promega)) or pEF1α-mIL4 by electroporation at either 0 V/cm, 1.2, kV/cm, or 1.33 kV/cm at 500 ug/ml. DNase was added to the transfected cells in transfection buffer 4 minutes following electroporation. The cells were incubated in transfection buffer with the DNase for 20 minutes in a 37° C. water bath and then cultured in complete culture medium without removing the DNase (addition of the culture medium resulted in a dilution of the DNase concentration of about 50-100×).

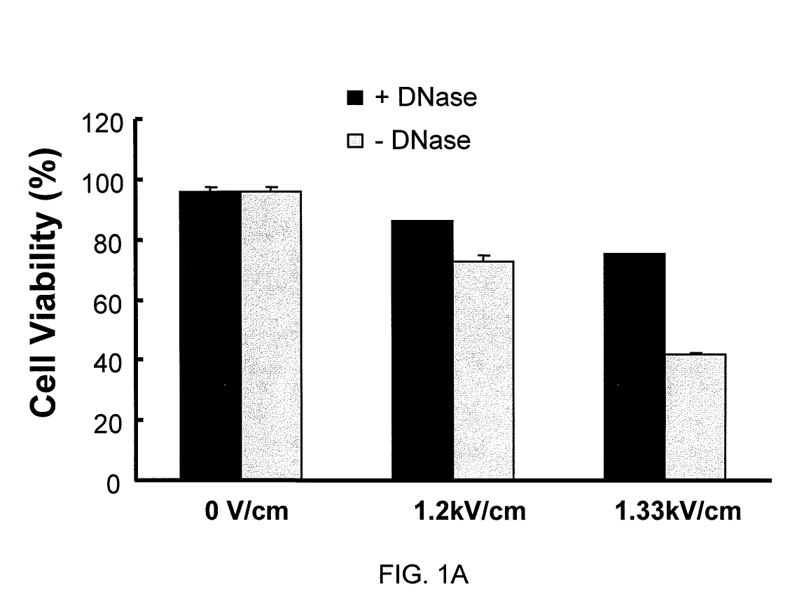

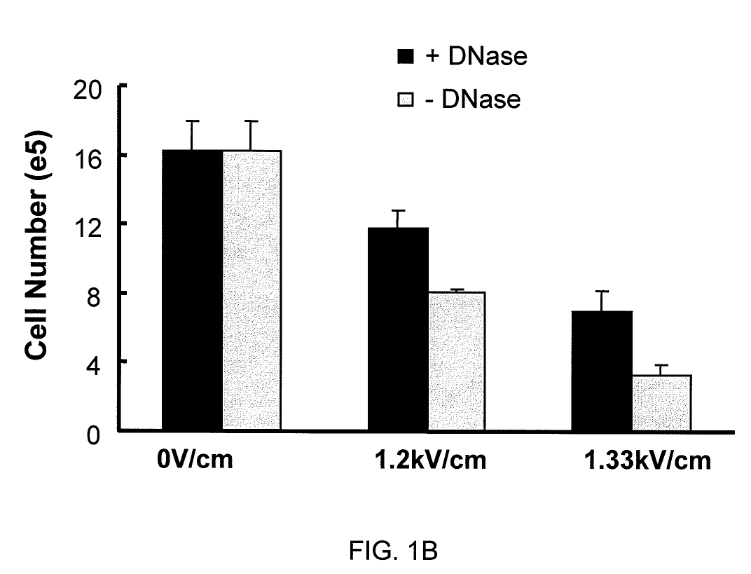

As shown in FIGS. 1A and 1B, there was no difference in cell viability in unelectroporated cells (0 V/cm) with or without DNase treatment. DNase treatment improved viability in the electroporated cells, with a more pronounced effect seen in cells receiving the highest input electrical energy (FIGS. 1A and 1B). Thus, post-EP DNase treatment improves Jurkat cell viability, and helps the cells tolerate higher electrical energy.

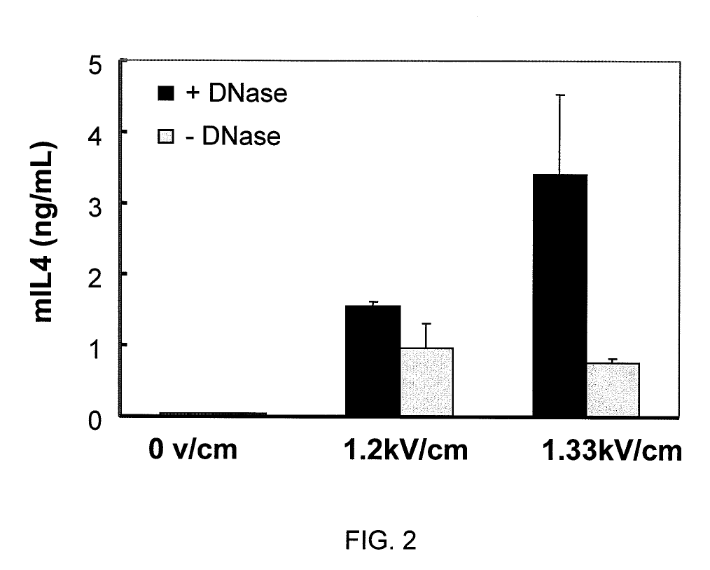

In addition to improving cell viability, DNase treatment also enhanced transgene expression. As shown in FIG. 2, there was no detectable mIL-4 produced by the unelectroporated cells (0 V/cm) with or without DNase treatment. DNase treatment increased the amount of mIL-4 secreted by the electroporated cells, with a more pronounced effect seen in cells receiving the highest input electrical energy (FIG. 2). Thus, post-EP DNase treatment improves transgene expression in Jurkat cells by helping the cells tolerate higher electrical energy.

### Example 2

### DNase Treatment Improves Cell Viability and Transfection Efficiency in Jurkat Cells by Allowing the Cells to Tolerate Higher DNA Concentrations

A DNase stock solution was prepared by reconstituting the lyophilized DNase in electroporation (EP) buffer at a concentration of 2000 U/mL. The DNase was added to the transfected cells in a ration of 1 volume DNase stock solution to 5 volumes of transfected cells.

Jurkat cells were transfected with the plasmid pCMV-eGFP by electroporation at 1.5 kV/cm. The plasmid was added at a concentration of 0 μg/ml, 50 μg/ml, 100 μg/ml, or 200 μg/ml. DNase was added to the transfected cells immediately following electroporation. The transfected cells were kept in the transfection buffer with the DNase for 20 minutes in a 37° C. water bath and then cultured in complete culture medium without removing the DNase (addition of the culture medium resulted in a dilution of the DNase concentration of about 50-100×).

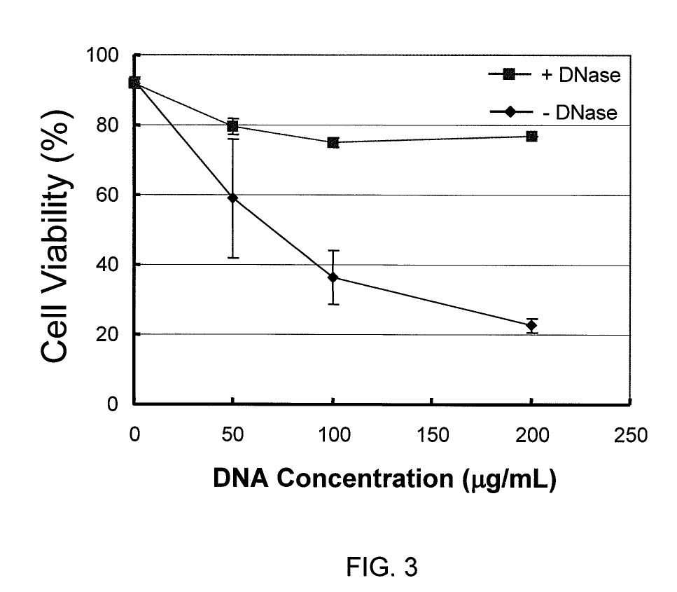

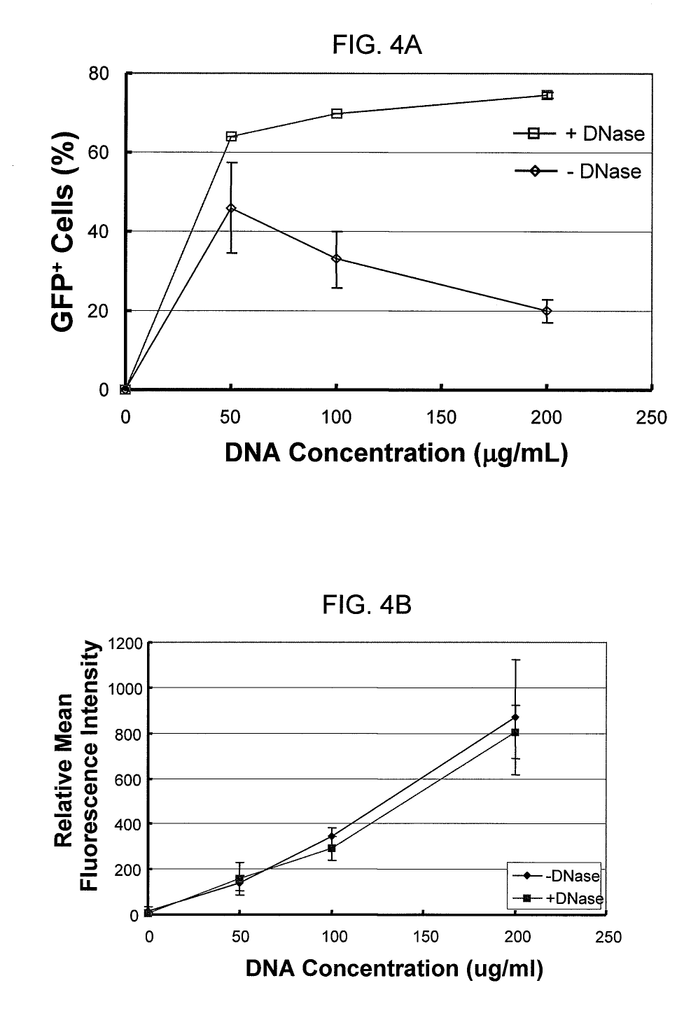

Cells were stained with propidium iodine and analyzed by FACS 2 days post transfection. As shown in FIG. 3, there was no difference in viability of cells electroporated in the absence of DNA with or without DNase treatment. For cells electroporated in the presence of DNA, DNase treatment significantly increased viability (FIG. 3). This effect was greatest at the highest DNA concentration tested. The percentage of GFP+ cells was also significantly increased by DNase treatment (FIG. 4A). Again, this effect was most pronounced at the highest DNA concentration tested. The mean fluorescence intensity of GFP-expressing cells was similar with or without DNase treatment (FIG. 4B). Thus, DNase treatment can increase transgene expression by allowing the target cells to tolerate a higher DNA concentration.

### Example 3

### Effect of Time Points for the DNase Treatment of Electroporated K562 Cells

A DNase stock solution was prepared by reconstituting the lyophilized DNase in electroporation (EP) buffer at a concentration of 2000 U/mL. The DNase was added to the transfected cells in a ration of 1 volume DNase stock solution to 10 volumes of transfected cells.

K562 cells were transfected with the plasmid pGEG-mIL-12 by electroporation at 1.5 kV/cm. The plasmid was added at a concentration of 175 μg/ml. DNase was added to the transfected cells at the time points indicated. The cells were incubated in transfection buffer with the DNase for 20 minutes in a 37° C. water bath and then cultured in complete culture medium without removing DNase (final DNase concentration in culture medium was diluted 50-100×).

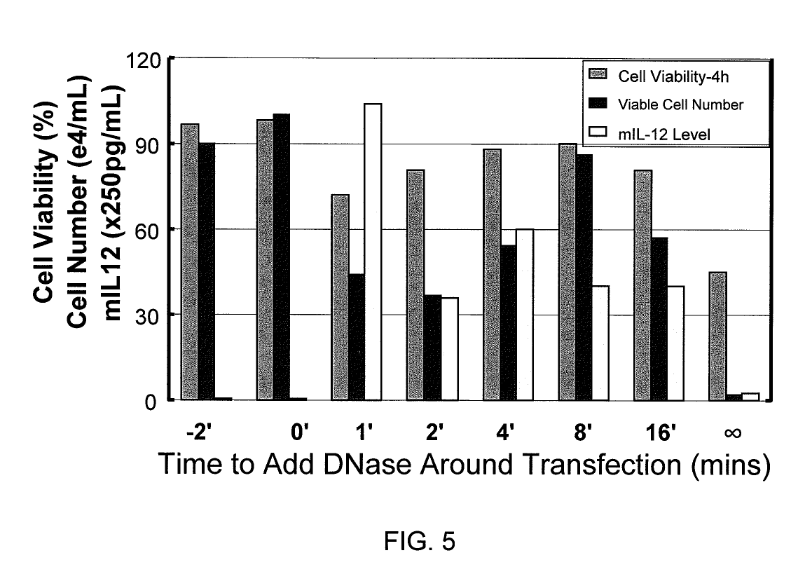

As shown in FIG. 5, adding the DNase to the cells 2 minutes prior to or during (−0′) electroporation resulted in undetectable transgene expression of mIL-12, presumably due to the degradation of the plasmid before it could be taken up by the cells. mIL-12 transgene expression was highest when DNase was added post-EP (FIG. 5). Cell viability, total cell numbers, and mIL-12 all declined when DNase was not added post-EP (∞ sample in the figure).

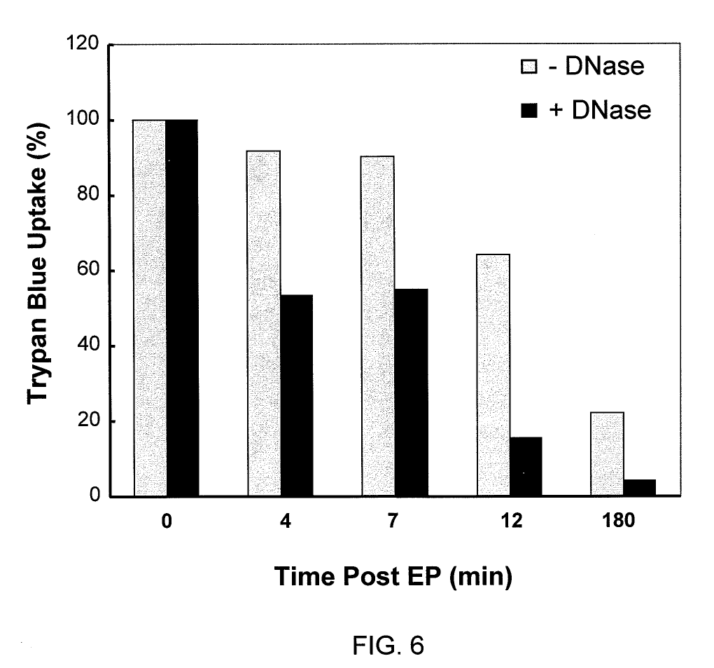

As shown in FIG. 6, DNase helps K562 cell membrane recovery following electroporation as assayed by Trypan blue uptake. DNase was added to pCMV-eGFP transfected K562 cells at 1 min after EP. The transfected cells were analyzed by trypan blue staining. DNase treated cells (darker column) took up significantly less trypan blue than untreated cells when analying at 4, 7 12, 180 minutes post EP.

### Example 4

### DNase Treatment Reduces Immediate Necrosis of Electroporated Cells

A DNase stock solution was prepared by reconstituting the lyophilized DNase in electroporation (EP) buffer at a concentration of 2000 U/mL. The DNase was added to the transfected cells in a ration of 1 volume DNase stock solution to 10 volumes of transfected cells.

K562 cells were transfected with either a backbone plasmid (3 kb in length), the GFP marker gene plasmid (300 μg/ml, pCMV-eGFP, 5 kb), pGAG-mIL12, (a plasmid carrying mIL12 transgene, 175 μg/ml, 13 kb in length), or pGAG-Endo-IRES-Angio, (a plasmid carrying dual transgenes, human endotatin and angiostatin, 13 kb in length 175 μg/ml) by electroporation at 1.5 kV/cm. The cells and DNA were mixed for either 0 minutes or 20 minutes prior to electroporation. DNase was added to the transfected cells 1-4 min following electroporation. The transfected cells were incubated in transfection buffer with the DNase for 20 min in a 37° C. water bath and then cultured in complete culture medium without removing DNase (final DNase concentration in culture medium was diluted 50-100×).

FACS analysis was used to assess the number of viable cells 3 hours and 24 hours post-EP. The data presented in Tables 4 and 5, below, demonstrate that DNase treatment significantly increased the percentage of viable K562 cells when the cells were transfected with large plasmids.

TABLE 4
FACS analysis 3 h post EP

| | | EP at 0 min post mixing | | | | | | EP at 20 min post mixing | | | | | |
|---|---|---|---|---|---|---|---|---|---|---|---|---|---|
| | | No DNase | | | With DNase | | | No DNase | | | With DNase | | |
| Plasmid DNA | size (Kb) | V | GFP + | MFI | V | GFP + | MFI | V | GFP + | MFI | V | GFP + | MFI |
| Plasmid Backbone | 3 | 76 | N/A | N/A | 82 | N/A | N/A | 60 | N/A | N/A | 63 | N/A | N/A |
| GFP Plasmid | 5 | 87 | 77 | 986 | 88 | 77 | 853 | 77 | 74 | 1026 | 80 | 78 | 1234 |
| DO15 | 13 | 30 | N/A | N/A | 55 | N/A | N/A | 14 | N/A | N/A | 35 | N/A | N/A |
| DO24 | 13 | 24 | N/A | N/A | 67 | N/A | N/A | 20 | N/A | N/A | 35 | N/A | N/A |

TABLE 5
FACS analysis 24 h post EP

| | | EP at 0 min post mixing | | | | | | EP at 20 min post mixing | | | | | |
|---|---|---|---|---|---|---|---|---|---|---|---|---|---|
| | | No DNase | | | With DNase | | | No DNase | | | With DNase | | |
| Plasmid DNA | size (kb) | V (%) | GFP + (%) | MFI | V (%) | GFP + (%) | MFI | V (%) | GFP + (%) | MFI | V (%) | GFP + | MFI |
| GFP Plasmid | 5 | 87 | 99 | 7055 | 87 | 99 | 6751 | 77 | 97 | 7239 | 85 | 99 | 7433 |
| DO24 | 13 | 13 | N/A | N/A | 70 | N/A | N/A | 10 | N/A | N/A | 70 | N/A | N/A |

The data presented in Table 6, below, further demonstrate that DNase treatment improves the viability of K562 cells when the cells were transfected with larger plasmids. In this assay, viability is assayed by the number of viable cells recovered 24 hours post-EP.

TABLE 6
Recovered cells 24 h post EP (e6)

| Plasmid | Size | EP at 0 min post mixing | | EP at 20 min post mixing | |
|---|---|---|---|---|---|
| DNA | (kb) | No DNase | With DNase | No DNase | With DNase |
| GFP | 5 | 5.2 | 5.1 | 3.7 | 3.3 |
| Plasmid | | | | | |
| DO24 | 13 | 0.3 | 1.6 | 0.25 | 2.4 |

### Example 5

### DNase Treatment Improves Viability and Transgene Expression in 293T Cells

A DNase stock solution was prepared by reconstituting the lyophilized DNase in electroporation (EP) buffer at a concentration of 2000 U/mL. The DNase was added to the transfected cells in a ration of 1 volume DNase stock solution to 10 volumes of transfected cells.

293T cells were transfected with the plasmid pGAG-mILL2, by electroporation at 1.5 kV/cm. The plasmid was added at a concentration of 175 μg/ml. DNase was added to the transfected cells immediately after electroporation. The transfected cells were incubated in transfection buffer with the DNase for 20 minutes in a 37° C. water bath and then cultured in full culture medium without removing the DNase (final DNase concentration in culture medium was diluted 50-100×).

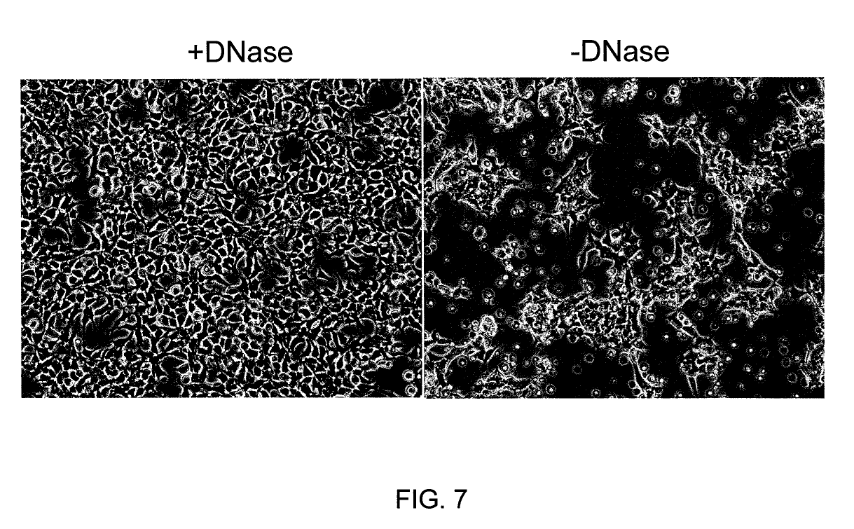

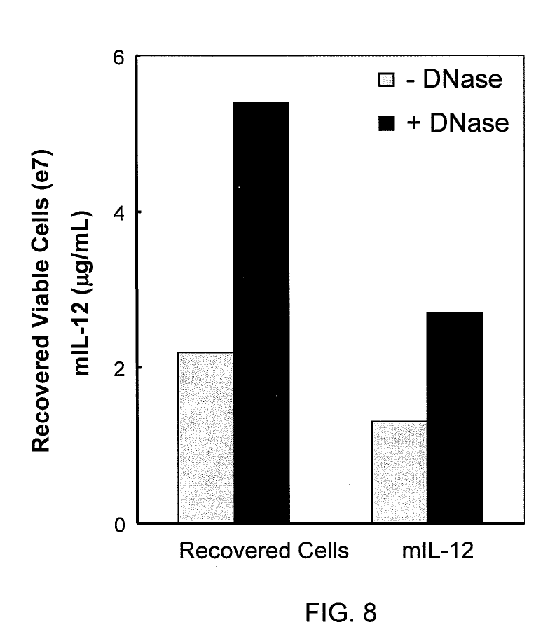

Analysis of the cell culture plates 16 hours and 24 hours post-EP revealed that significantly more DNase treated 293T cells survived electroporation than untreated 293T cells (FIGS. 7 and 8). At 24 hours post-EP, the number of viable cells in the DNase-treated population was more than 2-fold greater than the number of viable cells in the untreated population. The DNase treated cell population also secreted significantly more mIL-12 at 24 hours post-EP than did the untreated cells (FIG. 8).

### Example 6

### Viral Vector Production

Post-EP DNase treatment enhanced viral vector production in 293T cells and suspension K562 cells. A DNase stock solution was prepared by reconstituting the lyophilized DNase in electroporation (EP) buffer at a concentration of 2000 U/mL. The DNase was added to the transfected cells in a ratio of 1 volume DNase stock solution to 10 volumes of transfected cells.

Suspension K562 cells were transfected with three plasmids encoding packaging signal-eGFP, gag/pol, and VSV G, respectively, by electroporation at either 1.1, 1.3, or 1.5 kV/cm. The plasmids were added at a total concentration of 310 μg/ml. DNase was added to the transfected cells immediately after electroporation. The transfected cells were incubated in the transfection buffer with the DNase for 20 minutes in a 37° C. water bath and then cultured in full culture medium without removing the DNase (final DNase concentration in culture medium was diluted 50-100×).

The data in Table 7, below, show that DNase treatment significantly increased lentiviral vector titers in K562 cells that received the highest input energy during electroporation.

TABLE 7

| Electric | Viral Vector Titre (x1e5 TU/ml) | | | |
|---|---|---|---|---|
| Field | EP immediately after mixing | | EP 20 min after mixing | |
| (kV/cm) | No DNase | With DNase | No DNase | With DNase |
| 1.1 | 1.1 | 1 | 1.5 | 2 |
| 1.3 | 4.6 | 5.5 | 2.4 | 6.8 |
| 1.5 | 5.2 | 9.6 | 2.8 | 4.3 |

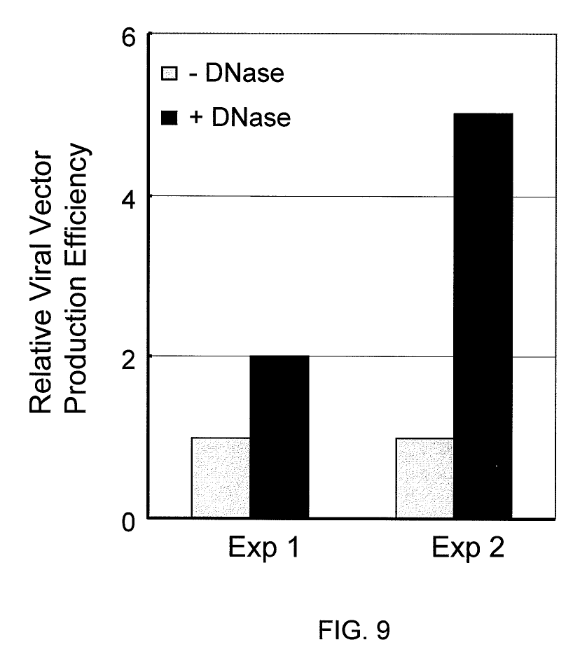

In another set of studies, 293T cells were transfected with the same three plasmids described in the experiment above, by electroporation at approximately 1 kV/cm. The plasmids were added at a concentration of approximately 310 μg/ml. DNase was added to the transfected cells immediately after electroporation. The transfected cells were incubated in the transfection buffer with the DNase for 20 minutes in a 37° C. water bath and then cultured in complete culture medium without removing DNase (final DNase concentration in culture medium was diluted 50-100×). As shown in FIG. 9, DNase treatment significantly increased lentiviral vector titers in 293T cells.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,352,883
U.S. Pat. No. 4,683,202
U.S. Pat. No. 5,302,523
U.S. Pat. No. 5,322,783
U.S. Pat. No. 5,464,765
U.S. Pat. No. 5,538,877
U.S. Pat. No. 5,538,880
U.S. Pat. No. 5,550,318
U.S. Pat. No. 5,563,055
U.S. Pat. No. 5,610,042
U.S. Pat. No. 5,612,207
U.S. Pat. No. 5,720,921
U.S. Pat. No. 5,789,215
U.S. Pat. No. 5,925,565
U.S. Pat. No. 5,928,906
U.S. Pat. No. 5,935,819
U.S. Pat. No. 6,074,605
U.S. Pat. No. 6,074,605
U.S. Pat. No. 6,090,617
U.S. Pat. No. 6,090,617
U.S. Pat. No. 6,485,961
U.S. Pat. No. 6,617,154
U.S. Pat. No. 6,773,669
U.S. patent Ser. No. 10/080,272
U.S. patent Ser. No. 10/225,446
U.S. patent Ser. No. 10/675,592
U.S. patent Ser. No. 10/751,586
U.S. patent Ser. No. 10/781,440
Almendro et al., J. Immunol., 157(12):5411-5421, 1996.
Angel et al., Cell, 49:729, 1987b.
Angel et al., Mol. Cell. Biol., 7:2256, 1987a.
Atchison and Perry, Cell, 46:253, 1986.
Atchison and Perry, Cell, 48:121, 1987.
Ausubel et al., In: Current Protocols in Molecular Biology, John, Wiley & Sons, Inc, New York, 1996.
Banerji et al., Cell, 27(2 Pt 1):299-308, 1981.
Banerji et al., Cell, 33(3):729-740, 1983.
Berkhout et al., Cell, 59:273-282, 1989.
Blanar et al, EMBO J., 8:1139, 1989.
Bodine and Ley, EMBO J, 6:2997, 1987.
Boshart et al., Cell, 41:521, 1985.
Boshart et al., Cell, 41:521, 1985.
Bosze et al, EMBO J., 5(7):1615-1623, 1986.
Braddock et al., Cell, 58:269, 1989.
Bulla and Siddiqui, J. Virol., 62:1437, 1986.
Campbell and Villarreal, Mol. Cell. Biol., 8:1993, 1988.
Campere and Tilghman, Genes and Dev., 3:537, 1989.
Campo et al., Nature, 303:77, 1983.
Carbonelli et al., FEMS Microbiol. Lett., 177(1):75-82, 1999.
Celander and Haseltine, J. Virology, 61:269, 1987.
Celander et al., J. Virology, 62:1314, 1988.
Chandler et al., Cell, 33:489, 1983.
Chandler et al., Proc. Natl. Acad. Sci. USA, 94(8):3596-601, 1997.
Chang et al., Mol. Cell. Biol., 9:2153, 1989.
Chatterjee et al., Proc. Natl. Acad. Sci. USA, 86:9114, 1989.
Chen and Okayama, Mol. Cell Biol., 7(8):2745-2752, 1987.
Choi et al., Cell, 53:519, 1988.
Christensen and Quail, Transgenic Res., 5(3):213-218, 1996.
Cocea, Biotechniques, 23(5):814-816, 1997.
Cohen et al., J. Cell. Physiol., 5:75, 1987.
Costa et al., Mol. Cell. Biol., 8:81, 1988.
Cripe et al., EMBO J., 6:3745, 1987.
Cristiano et al., Cancer Detect. Prev., 22(5):445-454, 1998.
Culotta and Hamer, Mol. Cell. Biol., 9:1376, 1989.
Dandolo et al., J. Virology, 47:55-64, 1983.
De Villiers et al., Nature, 312(5991):242-246, 1984.
Deschamps et al., Science, 230:1174-1177, 1985.
Edbrooke et al., Mol. Cell. Biol., 9:1908, 1989.
Edlund et al., Science, 230:912-916, 1985.
Eisenstein and Munro, Enzyme, 44(1-4):42-58, 1990.
Fechheimer, et al., Proc Natl. Acad. Sci. USA, 84:8463-8467, 1987.
Feng and Holland, Nature, 334:6178, 1988.
Firak and Subramanian, Mol. Cell. Biol., 6:3667, 1986.
Foecking and Hofstetter, Gene, 45(1):101-105, 1986.
Fraley et al., Proc. Natl. Acad. Sci. USA, 76:3348-3352, 1979.
Fujita et al., Cell, 49:357, 1987.
Gabriel and Teissie, Biophys. J., 76(4):2158-2165, 1999.
Gagne et al., Mol. Reprod. Dev., 29(1):6-15, 1991.
Gagne et al., Mol. Reprod. Dev., 29(1):6-15, 1991.
Gilles et al., Cell, 33:717, 1983.
Gloss et al., EMBO J., 6:3735, 1987.
Godbout et al., Mol. Cell. Biol., 8:1169, 1988.
Goodboum and Maniatis, Proc. Natl. Acad. Sci. USA, 85:1447, 1988.
Goodbourn et al., Cell, 45:601, 1986.
Gopal, Mol. Cell Biol., 5:1188-1190, 1985.
Graham and Van Der Eb, Virology, 52:456-467, 1973.
Greene et al., Immunology Today, 10:272, 1989
Grosschedl and Baltimore, Cell, 41:885, 1985.
Guo et al., Gene Ther., 3(9):802-810, 1996.
Harland and Weintraub, J. Cell Biol., 101(3):1094-1099, 1985.
Haslinger and Karin, Proc. Natl. Acad. Sci. USA, 82:8572, 1985.
Hauber and Cullen, J. Virology, 62:673, 1988.
Hen et al., Nature, 321:249, 1986.
Hensel et al., Lymphokine Res., 8:347, 1989.
Herr and Clarke, Cell, 45:461, 1986.
Hibino et al., Biophys. J., 64(6):1789-1800, 1993.
Hirochika et al., J. Virol., 61:2599, 1987.
Hirsch et al., Mol. Cell. Biol., 10: 1959, 1990.
Holbrook et al., Virology, 157:211, 1987.
Horlick and Benfield, Mol. Cell. Biol., 9:2396, 1989.
Huang et al., Cell, 27:245, 1981.
Hug et al., Mol. Cell. Biol., 8:3065, 1988.
Hwang et al., Mol. Cell. Biol., 10:585, 1990.
Imagawa et al., Cell, 51:251, 1987.
Imbra and Karin, Nature, 323:555, 1986.
Imler et al, Mol. Cell. Biol., 7:2558, 1987.
Imperiale and Nevins, Mol. Cell. Biol., 4:875, 1984.
Jakobovits et al., Mol. Cell. Biol., 8:2555, 1988.
Jameel and Siddiqui, Mol. Cell. Biol., 6:710, 1986.
Jaynes et al., Mol. Cell. Biol., 8:62, 1988.
Johnson et al., Mol. Cell. Biol., 9:3393, 1989.
Kadesch and Berg, Mol. Cell. Biol., 6:2593, 1986.
Kaeppler et al., Plant Cell Reports, 9:415-418, 1990.
Kakorin et al., Biophys. Chem., 58(1-2):109-116, 1996.
Kaneda et al., Science, 243:375-378, 1989.
Karin et al., Mol. Cell. Biol., 7:606, 1987.
Karin et al., Mol. Cell. Biol., 7:606, 1987.
Katinka et al., Cell, 20:393, 1980.
Kato et al, J. Biol. Chem., 266:3361-3364, 1991.
Kawamoto et al., Mol. Cell. Biol., 8:267, 1988.
Kemppainen et al., In: DNA Removal for Biprocessing Applications, Ambion TechNotes, Vol. 11:5, 20042004
Kiledjian et al., Mol. Cell. Biol., 8:145, 1988.
Kim et al., Gene, 91(2):217-223, 1990.
Kitamura and Komiyama, Nucl. Acids Res., 30(19):1-6, 2002.
Klamut et al., Mol. Cell. Biol., 10:193, 1990.
Koch et al., Mol. Cell. Biol., 9:303, 1989.
Kotnik et al., Bioenerg., 43:281-291; 45:3-16, 1998.
Kraus et al. FEBS Lett., 428(3):165-170, 1998.
Kriegler and Botchan, In: Eukaryotic Viral Vectors, Gluzman (Ed.), Cold Spring Harbor: Cold Spring Harbor Laboratory, NY, 1982.
Kriegler and Botchan, Mol. Cell. Biol., 3:325, 1983.
Kriegler et al., Cell, 38:483, 1984.
Kriegler et al., Cell, 53:45, 1988.
Kuhl et al., Cell, 50:1057, 1987.
Kunz et al., Nucl. Acids Res., 17:1121, 1989.
Lareyre et al., J. Biol. Chem., 274(12):8282-8290, 1999.
Larsen et al., Proc Natl. Acad. Sci. USA., 83:8283, 1986.
Laspia et al., Cell, 59:283, 1989.
Latimer et al., Mol. Cell. Biol., 10:760, 1990.
Lee et al., Biochem. Biophys. Res. Commun., 238(2):462-467, 1997.
Lee et al, Nature, 294:228, 1981.
Lee et al., Nucleic Acids Res., 12:4191-206, 1984.
Levenson et al., Hum. Gene Ther., 9(8):1233-1236, 1998.
Levinson et al., Nature, 295:79, 1982.
Lin et al., Mol. Cell. Biol., 10:850, 1990.
Luria et al, EMBO J., 6:3307, 1987.
Lusky and Botchan, Proc. Natl. Acad. Sci. USA, 83:3609, 1986.
Lusky et al., Mol. Cell. Biol., 3:1108, 1983.
Macejak and Sarnow, Nature, 353:90-94, 1991.
Majors and Varmus, Proc. Natl. Acad. Sci. USA, 80:5866, 1983.
McNeall et al., Gene, 76:81, 1989.
Miksicek et al., Cell, 46:203, 1986.
Mir, Bioelectrochem., 53:1-10, 2000.
Miyake et al., J. Cell Science, 114(19):3487-3494, 2001.
Mizrahi, Dev. Biol. Stand., 55:219-230, 1983.
Mordacq and Linzer, Genes and Dev., 3:760, 1989.
Moreau et al., Nucl. Acids Res., 9:6047, 1981.
Moreau et al., Nucl. Acids Res., 9:6047, 1981.
Muesing et al., Cell, 48:691, 1987.
Neumann et al., Proc. Natl. Acad. Sci. USA, 96(16):9345-9350, 1999.
Ng et al., Nuc. Acids Res., 17:601, 1989.
Nicolau and Sene, Biochim. Biophys. Acta, 721:185-190, 1982.
Nicolau et al., Methods Enzymol., 149:157-176, 1987.
Nomoto et al., Gene, 236(2):259-271, 1999.
Okabe et al., FEBS Lett., 407(3):313-319, 1997.
Ondek et al., EMBO J., 6:1017, 1987.
Omitz et al., Mol. Cell. Biol., 7:3466, 1987.
Palmiter et al., Nature, 300:611, 1982.
PCT Appln. WO 03/018751
PCT Appln. WO 03/018751
PCT Appln. WO 2004/031353
PCT Appln. WO 2004/031353
PCT Appln. WO 94/09699
PCT Appln. WO 94/17178
PCT Appln. WO 95/06128
Pech et al., Mol. Cell. Biol., 9:396, 1989.
Pelletier and Sonenberg, Nature, 334(6180):320-325, 1988.
Perez-Stable and Constantini, Mol. Cell. Biol., 10:1116, 1990.
Phillips et al., In: Large Scale Mammalian Cell Culture, Feder and Tolbert (Eds.), Academic Press, Orlando, Fla., U.S.A., 1985.
Picard and Schaffner, Nature, 307:83, 1984.
Pinkert et al., Genes and Dev., 1:268, 1987.
Ponta et al., Proc. Natl. Acad. Sci. USA, 82:1020, 1985.
Porton et al., Mol. Cell. Biol., 10:1076, 1990.
Queen and Baltimore, Cell, 35:741, 1983.
Quinn et al., Mol. Cell. Biol., 9:4713, 1989.
Redondo et al., Science, 247:1225, 1990.
Reisman and Rotter, Mol. Cell. Biol., 9:3571, 1989.
Resendez Jr. et al., Mol. Cell. Biol., 8:4579, 1988.
Ripe et al., Mol. Cell. Biol., 9:2224, 1989.
Rippe, et al., Mol. Cell Biol., 10:689-695, 1990.
Rittling et al., Nuc. Acids Res., 17:1619, 1989.
Robbins and Ghivizzani, Pharmacol Ther, 80(1):35-47, 1998.
Rols and Teissie, Biophys. J., 75(3):1415-1423, 1998.
Rosen et al., Cell, 41:813, 1988.
Sakai et al., Genes and Dev., 2:1144, 1988.
Sambrook et al., In: Molecular cloning, Cold Spring Harbor Laboratory Press, Cold Spring Harbor, N.Y., 2001.
Satake et al., J. Virology, 62:970, 1988.
Schaffner et al., J. Mol. Biol., 201:81, 1988.
Searle et al., Mol. Cell. Biol., 5:1480, 1985.
Sharp and Marciniak, Cell, 59:229, 1989.
Shaul and Ben-Levy, EMBO J., 6:1913, 1987.
Sherman et al., Mol. Cell. Biol., 9:50, 1989.
Singer-Sam et al., Gene, 32(3):409-417, 1984.
Sleigh and Lockett, J. EMBO, 4:3831, 1985.
Spalholz et al., Cell, 42:183, 1985.
Spandau and Lee, J. Virology, 62:427, 1988.
Spandidos and Wilkie, EMBO J., 2:1193, 1983.
Stephens and Hentschel, Biochem. J., 248:1, 1987.
Stuart et al., Nature, 317:828, 1985.
Sullivan and Peterlin, Mol. Cell. Biol., 7:3315, 1987.
Swartzendruber and Lehman, J. Cell. Physiology, 85:179, 1975.
Takebe et al., Mol. Cell Biol., 8(1):466-472, 1988.
Takebe et al., Mol. Cell. Biol., 8:466, 1988.
Tavernier et al., Nature, 301:634, 1983.
Taylor and Kingston, Mol. Cell. Biol., 10:165, 1990a.
Taylor and Kingston, Mol. Cell. Biol., 10: 176, 1990b.
Taylor et al., J. Biol. Chem., 264:15160, 1989.
Thiesen et al., J. Virology, 62:614, 1988.
Treisman, Cell, 42:889, 1985.
Tronche et al., Mol. Biol. Med., 7:173, 1990.
Trudel and Constantini, Genes and Dev., 6:954, 1987.
Tsumaki et al., J. Biol. Chem., 273(36):22861-22864, 1998.
Tyndell et al., Nuc. Acids. Res., 9:6231, 1981.
Van Wezel, Nature, 216(110):64-65, 1967.
Vannice and Levinson, J. Virology, 62:1305, 1988.
Vasseur et al., Proc Natl. Acad. Sci. USA, 77:1068, 1980.
Wang and Calame, Cell, 47:241, 1986.
Wang et al., Am. J. Physiol., 263(4 Pt 1):G480-486, 1992.
Wang et al., FEBS Letters, 353:138-142, 1994.
Wang et al., Science, 259(5098):1173-1175, 1993.
Weaver and Chizmadzhev, Bioenerg, 41:135-160, 1996.
Weber et al., Cell, 36:983, 1984.
Weinberger et al. Mol. Cell. Biol., 8:988, 1984.
Winoto and Baltimore, Cell, 59:649, 1989.
Wong et al., Gene, 10:87-94, 1980.
Wu et al., J. Exp. Med., 185:1681-1691, 1997.
Yamamoto et al., Nucl. Acids Res., 31(15):1-6, 2003.
Yamamoto et al., Nucl. Acids. Res., 32(19):1-7, 2004.
Yew et al., Mol. Ther., 4(1):75-82, 2001.
Yutzey et al. Mol. Cell. Biol., 9:1397, 1989.
Zhao-Emonet et al., Biochim. Biophys. Acta, 1442(2-3):109-119, 1998.

What is claimed is:

1. A method for increasing viability of a transfected cell, the method comprising:
(a) transfecting a cell with a nucleic acid sequence; and
(b) contacting the transfected cell with a nucleic acid digesting agent under conditions effective to increase the viability of the transfected cell relative to the viability of a control transfected cell not contacted with the nucleic acid digesting agent.

2. The method of claim 1, wherein the nucleic acid digesting agent is a nuclease.

3. The method of claim 2, wherein the nuclease is a DNase.

4. The method of claim 2, wherein the nuclease is a restriction endonuclease.

5. The method of claim 1, wherein the nucleic acid sequence is a DNA sequence.

6. The method of claim 1, wherein the nucleic acid sequence is a RNA sequence.

7. The method of claim 1, wherein the transfection comprises electroporation.

8. The method of claim 1, wherein the transfected cell is contacted with the nuclease between 0-60 minutes after transfection.

9. The method of claim 1, wherein the nucleic acid sequence is an expression vector.

10. The method of claim 1, wherein the nucleic acid sequence is greater than about 5 kb in length.

11. The method of claim 1, wherein the nucleic acid sequence is greater than about 10 kb in length.

12. The method of claim 1, wherein the nucleic acid sequence is greater than about 12 kb in length.

13. The method of claim 1, wherein the nucleic acid sequence is greater than about 13 kb in length.

14. The method of claim 9, wherein the expression vector encodes a protein.

15. The method of claim 14, wherein the protein is a cytokine.

16. The method of claim 9, wherein the expression vector encodes one or more viral genes.

17. A method for increasing transfection efficiency in a population of transfected cells, the method comprising:
(a) transfecting the cells with a nucleic acid sequence; and
(b) contacting the transfected cells with a nuclease under conditions effective to increase the transfection efficiency in the population of transfected cells relative to the transfection efficiency in a control population of transfected cells not contacted with the nuclease.

18. The method of claim 17, wherein increasing the transfection efficiency in a population of cells is further defined as increasing the percentage of viable, transfected cells in the population.

19. The method of claim 17, wherein increasing the transfection efficiency in a population of cells is further defined as increasing the percentage of transfected cells in the population.

20. The method of claim 17, wherein increasing the transfection efficiency in a population of cells is further defined as increasing the expression level of a transgene in the transfected cells in the population.

21. A method for increasing viability of a cell after electroporation, the method comprising:
(a) transfecting a cell with a nucleic acid sequence by electroporation; and
(b) contacting the cell with a nuclease after electroporation under conditions effective to increase the viability of the transfected cell relative to the viability of a control electroporated cell not contacted with the nuclease.

22. The method of claim 21, further comprising incubating the cell in electroporation buffer after electroporation.

23. The method of claim 22, wherein the cell is incubated in the electroporation buffer for about 0-20 minutes.

24. The method of claim 22, wherein the nuclease is added to the electroporation buffer.

25. The method of claim 21, wherein the electroporation is static electroporation.

26. The method of claim 21, wherein the electroporation is flow electroporation.

27. The method of claim 21, wherein the electroporation is streaming electroporation.

28. The method of claim 21, wherein the electroporation is variable flow electroporation.

29. The method of claim 21, wherein the cell is contacted with the nuclease between 0-60 minutes after electroporation.

30. The method of claim 29, wherein the cell is contacted with the nuclease between 0-16 minutes after electroporation.
